SEQUENCE LISTING (108 sequences)

SEQ ID NO: 1 - DNA, 676 bp, Artificial Sequence
  Synthetic Oligonucleotide
ggtaccgagctcggatccactagtaacggccgccagtgtgctggaattgcccttaagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgggtcttcgagaagacctgttttagagctagaaatagcaagttaaaataaggctagtccgttatcaacttgaaaaagtggcaccgagtcggtgctttttttgtcccttcgaagggcctttctcagatatccatcacactggcggccgctcgaggtcgacggtatcgataagctt

SEQ ID NO: 2 - DNA, 678 bp, Artificial Sequence
  Synthetic Oligonucleotide
ggtaccgagctcggatccactagtaacggccgccagtgtgctggaattgcccttaagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattnnnnnnnnnnnnnnnnnnnngttttagagctagaaatagcaagttaaaataaggctagtccgttatcaacttgaaaaagtggcaccgagtcggtgctttttttgtcccttcgaagggcctttctcagatatccatcacactggcggccgctcgaggtcgacggtatcgataagctt

SEQ ID NO: 3 - DNA, 474 bp, Artificial Sequence
  Synthetic Oligonucleotide
ggtaccgagctcggatccactagtaacggccgccagtgtgctggaattgcccttggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgtgggtcttcgagaagacctgttttagagctagaaatagcaagttaaaataaggctagtccgttatcaacttgaaaaagtggcaccgagtcggtgctttttttgtcccttcgaagggcaattctgcagatatccatcacactggcggccgctcgaggtcgacggtatcgataagctt

SEQ ID NO: 4 - DNA, 476 bp, Artificial Sequence
  Synthetic Oligonucleotide
ggtaccgagctcggatccactagtaacggccgccagtgtgctggaattgcccttggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgtnnnnnnnnnnnnnnnnnnnngttttagagctagaaatagcaagttaaaataaggctagtccgttatcaacttgaaaaagtggcaccgagtcggtgctttttttgtcccttcgaagggcaattctgcagatatccatcacactggcggccgctcgaggtcgacggtatcgataagctt

SEQ ID NO: 5 - DNA, 30 bp, Artificial Sequence
  Synthetic Oligonucleotide
acacgctcgagatggtgagcaagggcgagg

SEQ ID NO: 6 - DNA, 44 bp, Artificial Sequence
  Synthetic Oligonucleotide
acacggtcgacactagtggatccgtggcggatcttgaagttcac

SEQ ID NO: 7 - DNA, 44 bp, Artificial Sequence
  Synthetic Oligonucleotide
acacgggatccactagtgtcgacgaccacatgaagcagcacgac

SEQ ID NO: 8 - DNA, 29 bp, Artificial Sequence
  Synthetic Oligonucleotide
acacggagctcttacttgtacagctcgtc

SEQ ID NO: 9 - DNA, 31 bp, Artificial Sequence
  Synthetic Oligonucleotide
ttactcgagatggactataaggaccacgacg

SEQ ID NO: 10 - DNA, 32 bp, Artificial Sequence
  Synthetic Oligonucleotide
attggatccttactttttcttttttgcctggc

SEQ ID NO: 11 - DNA, 20 bp, Artificial Sequence
  Synthetic Oligonucleotide
aagcttcgttgaacaacgga

SEQ ID NO: 12 - DNA, 22 bp, Artificial Sequence
  Synthetic Oligonucleotide
cgaagggacaatcactacttcg

SEQ ID NO: 13 - DNA, 42 bp, Artificial Sequence
  Synthetic Oligonucleotide
ttattttaacttgctatttctagctctaaaacaggtcttctc

SEQ ID NO: 14 - DNA, 41 bp, Artificial Sequence
  Synthetic Oligonucleotide
gttaaaataaggctagtccgttatcaacttgaaaaagtggc

SEQ ID NO: 15 - DNA, 20 bp, Artificial Sequence
  Synthetic Oligonucleotide
ggatcatgaaccaacggcct

SEQ ID NO: 16 - DNA, 18 bp, Artificial Sequence
  Synthetic Oligonucleotide
aacacaagcgacagcgcg

SEQ ID NO: 17 - DNA, 42 bp, Artificial Sequence
  Synthetic Oligonucleotide
gccagtgtgctggaattgcccttggatcatgaaccaacggcc

SEQ ID NO: 18 - DNA, 45 bp, Artificial Sequence
  Synthetic Oligonucleotide
gctctaaaacaggtcttctcgaagacccacacaagcgacagcgcg

SEQ ID NO: 19 - DNA, 24 bp, Artificial Sequence
  Synthetic Oligonucleotide
gattgtgaacttcaagatccgcca

SEQ ID NO: 20 - DNA, 24 bp, Artificial Sequence
  Synthetic Oligonucleotide
aaactggcggatcttgaagttcac

SEQ ID NO: 21 - DNA, 24 bp, Artificial Sequence
  Synthetic Oligonucleotide
gattgtgggtcataacgatatctc

SEQ ID NO: 22 - DNA, 24 bp, Artificial Sequence
  Synthetic Oligonucleotide
aaacgagatatcgttatgacccac

SEQ ID NO: 23 - DNA, 24 bp, Artificial Sequence
  Synthetic Oligonucleotide
gattggacatacatgagctcctga

SEQ ID NO: 24 - DNA, 24 bp, Artificial Sequence
  Synthetic Oligonucleotide
aaactcaggagctcatgtatgtcc

SEQ ID NO: 25 - DNA, 24 bp, Artificial Sequence
  Synthetic Oligonucleotide
gattgtaagagctgacatagcctg

SEQ ID NO: 26 - DNA, 24 bp, Artificial Sequence
  Synthetic Oligonucleotide
aaaccaggctatgtcagctcttac

SEQ ID NO: 27 - DNA, 24 bp, Artificial Sequence
  Synthetic Oligonucleotide
gattgatgagcttctagctgttct

SEQ ID NO: 28 - DNA, 24 bp, Artificial Sequence
  Synthetic Oligonucleotide
aaacagaacagctagaagctcatc

SEQ ID NO: 29 - DNA, 24 bp, Artificial Sequence
  Synthetic Oligonucleotide
gtgtgcggagaacgacagccggtc

SEQ ID NO: 30 - DNA, 24 bp, Artificial Sequence
  Synthetic Oligonucleotide
aaacgaccggctgtcgttctccgc

SEQ ID NO: 31 - DNA, 21 bp, Artificial Sequence
  Synthetic Oligonucleotide
gaatctctgacgaatctatcc

SEQ ID NO: 32 - DNA, 20 bp, Artificial Sequence
  Synthetic Oligonucleotide
cactctttcttcatcccatc

SEQ ID NO: 33 - DNA, 20 bp, Artificial Sequence
  Synthetic Oligonucleotide
gatgggatgaagaaagagtg

SEQ ID NO: 34 - DNA, 20 bp, Artificial Sequence
  Synthetic Oligonucleotide
ctcatctctctaccaacaag

SEQ ID NO: 35 - DNA, 25 bp, Artificial Sequence
  Synthetic Oligonucleotide
tgttattagaagtggtagtggagtg

SEQ ID NO: 36 - DNA, 19 bp, Artificial Sequence
  Synthetic Oligonucleotide
agccgtcgctgtagtggtt

SEQ ID NO: 37 - DNA, 19 bp, Artificial Sequence
  Synthetic Oligonucleotide
ctttgggggcctctttgac

SEQ ID NO: 38 - DNA, 18 bp, Artificial Sequence
  Synthetic Oligonucleotide
atctgcgtgcggcgattc

SEQ ID NO: 39 - DNA, 5476 bp, Artificial Sequence
  2x35S-Cas9-Nos
aagcttgcatgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtgatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgacctcaacacaacatatacaaaacaaacgaatctcaagcaatcaagcattctacttctattgcagcaatttaaatcatttcttttaaagcaaaagcaattttctgaaaattttcaccatttacgaacgatactcgagatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaaggatcctgattgatcgatagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattc

SEQ ID NO: 40 - DNA, 479 bp, Artificial sequence
  pAtU6-chiRNA
aagcttcattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtagcgcgtgcgccaattctgcagacaaatggccccggg

SEQ ID NO: 41 - DNA, 5172 bp, Artificial sequence
  pAtUBQ-Cas9-tUBQ
cccgggatatttcacaaattgaacatagactacagaattttagaaaacaaactttctctctcttatctcacctttatcttttagagagaaaaagttcgatttccggttgaccggaatgtatctttgttttttttgttttgtaacatatttcgttttccgatttagatcggatctccttttccgttttgtcggaccttcttccggtttatccggatctaataatatccatcttagacttagctaagtttggatctgttttttggttagctcttgtcaatcgcctcatcatcagcaagaaggtgaaatttttgacaaataaatcttagaatcatgtagtgtctttggaccttgggaatgatagaaacgatttgttatagctactctatgtatcagaccctgaccaagatccaacaatctcataggttttgtgcatatgaaaccttcgactaacgagaagtggtcttttaatgagagagatatctaaaatgttatcttaaaagcccactcaaatctcaaggcataaggtagaaatgcaaatttggaaagtgggctgggccttttgtggtaaaggcctgtaacctagcccaatattagcaaaaccctagacgcgtacattgacatatataaacccgcctcctccttgtttagggtttctacgtgagagagacgaaacacaaaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaaggatccagagactcttatcaagaatcccatctcttgcttgcttttttttgttgcttccctttgatagggtttgtttttcttgtttcagtgactttctatgttaaaagataatgtcagtaaaaggatttggttttctattattctgaatcgattacggaagattcttgcttaattccaatctatacaagtatcgtgaaataatgaccgtttatgtggtacc

SEQ ID NO: 42 - DNA, 35 bp, Artificial Sequence
  Synthetic Oligonucleotide
gccaagcttcattcggagtttttgtatcttgtttc

SEQ ID NO: 43 - DNA, 41 bp, Artificial Sequence
  Synthetic Oligonucleotide
aatcactacttcgactctagctgtatataaactcagcttcg

SEQ ID NO: 44 - DNA, 38 bp, Artificial Sequence
  Synthetic Oligonucleotide
cgaagtagtgattgggtcttcgagaagacctgttttag

SEQ ID NO: 45 - DNA, 31 bp, Artificial Sequence
  Synthetic Oligonucleotide
tatcccggggccatttgtctgcagaattggc

SEQ ID NO: 46 - DNA, 37 bp, Artificial Sequence
  Synthetic Oligonucleotide
tggccccgggatatttcacaaattgaacatagactac

SEQ ID NO: 47 - DNA, 40 bp, Artificial Sequence
  Synthetic Oligonucleotide
ccttatagtccatggtttgtgtttcgtctctctcacgtag

SEQ ID NO: 48 - DNA, 33 bp, Artificial Sequence
  Synthetic Oligonucleotide
cacaaaccatggactataaggaccacgacggag

SEQ ID NO: 49 - DNA, 37 bp, Artificial Sequence
  Synthetic Oligonucleotide
tctggatccttactttttcttttttgcctggccggcc

SEQ ID NO: 50 - DNA, 40 bp, Artificial Sequence
  Synthetic Oligonucleotide
taaggatccagagactcttatcaagaatcccatctcttgc

SEQ ID NO: 51 - DNA, 43 bp, Artificial Sequence
  Synthetic Oligonucleotide
acggtaccacataaacggtcattatttcacgatacttgtatag

SEQ ID NO: 52 - DNA, 34 bp, Artificial Sequence
  Synthetic Oligonucleotide
gtggtacccattcggagtttttgtatcttgtttc

SEQ ID NO: 53 - DNA, 30 bp, Artificial Sequence
  Synthetic Oligonucleotide
acgaattcgccatttgtctgcagaattggc

SEQ ID NO: 54 - DNA, 21 bp, Artificial Sequence
  Synthetic Oligonucleotide
ggcgtctcttcttggaacatc

SEQ ID NO: 55 - DNA, 21 bp, Artificial Sequence
  Synthetic Oligonucleotide
ccgaaacatggtaacgagacc

SEQ ID NO: 56 - DNA, 20 bp, Artificial Sequence
  Synthetic Oligonucleotide
ggcgtctcttctcggaagat

SEQ ID NO: 57 - DNA, 20 bp, Artificial Sequence
  Synthetic Oligonucleotide
cggataaacaggtcttgcac

SEQ ID NO: 58 - DNA, 20 bp, Artificial Sequence
  Synthetic Oligonucleotide
atggtgatggctggtgcttc

SEQ ID NO: 59 - DNA, 22 bp, Artificial Sequence
  Synthetic Oligonucleotide
catgtaagcacacatgtgtggg

SEQ ID NO: 60 - DNA, 21 bp, Artificial Sequence
  Synthetic Oligonucleotide
ctgcccgtccatctaacctac

SEQ ID NO: 61 - DNA, 20 bp, Artificial Sequence
  Synthetic Oligonucleotide
gacttcgaccaccacgatgt

SEQ ID NO: 62 - DNA, 24 bp, Artificial Sequence
  Synthetic Oligonucleotide
gattgcccccatttgcttcaggcc

SEQ ID NO: 63 - DNA, 24 bp, Artificial Sequence
  Synthetic Oligonucleotide
aaacggcctgaagcaaatgggggc

SEQ ID NO: 64 - DNA, 24 bp, Artificial Sequence
  Synthetic Oligonucleotide
gattggacattcataacagagaca

SEQ ID NO: 65 - DNA, 24 bp, Artificial Sequence
  Synthetic Oligonucleotide
aaactgtctctgttatgaatgtcc

SEQ ID NO: 66 - DNA, 24 bp, Artificial Sequence
  Synthetic Oligonucleotide
gattgagagagctgatggacctgc

SEQ ID NO: 67 - DNA, 24 bp, Artificial Sequence
  Synthetic Oligonucleotide
aaacgcaggtccatcagctctctc

SEQ ID NO: 68 - DNA, 24 bp, Artificial Sequence
  Synthetic Oligonucleotide
gattgaggcgacaagtcgacaatt

SEQ ID NO: 69 - DNA, 24 bp, Artificial Sequence
  Synthetic Oligonucleotide
aaacaattgtcgacttgtcgcctc

SEQ ID NO: 70 - DNA, 20 bp, Artificial Sequence
  Synthetic Oligonucleotide
gattggagaccgaggtctct

SEQ ID NO: 71 - DNA, 20 bp, Artificial Sequence
  Synthetic Oligonucleotide
aaacagagacctcggtctcc

SEQ ID NO: 72 - DNA, 24 bp, Artificial Sequence
  Synthetic Oligonucleotide
gattggggtagggttcaattgaag

SEQ ID NO: 73 - DNA, 23 bp, Artificial Sequence
  Synthetic Oligonucleotide
aaaccttcaattgaaccctaccc

SEQ ID NO: 74 - DNA, 24 bp, Artificial Sequence
  Synthetic Oligonucleotide
gattgtgaagttaccaagaatcag

SEQ ID NO: 75 - DNA, 23 bp, Artificial Sequence
  Synthetic Oligonucleotide
aaacctgattcttggtaacttca

SEQ ID NO: 76 - DNA, 29 bp, Artificial Sequence
  Synthetic Oligonucleotide
ttacccgggaacacgaagtcacaaaaccc

SEQ ID NO: 77 - DNA, 33 bp, Artificial Sequence
  Synthetic Oligonucleotide
ggtctcccatggtgatgatgatcttcttctcgg

SEQ ID NO: 78 - DNA, 43 bp, Artificial Sequence
  Synthetic Oligonucleotide
aatggatccgtttgtttgttttttaatcgttttcatcaacatg

SEQ ID NO: 79 - DNA, 26 bp, Artificial Sequence
  Synthetic Oligonucleotide
aatggtaccacgagaacgtgctgagc

SEQ ID NO: 80 - DNA, 21 bp, Artificial Sequence
  Synthetic Oligonucleotide
ggttcataccaaagtctgagc

SEQ ID NO: 81 - DNA, 22 bp, Artificial Sequence
  Synthetic Oligonucleotide
tcaagtagtcaacttaaggggg

SEQ ID NO: 82 - DNA, 33 bp, Artificial Sequence
  Synthetic Oligonucleotide
catggtctcacgccatcgtgcctcagagctttc

SEQ ID NO: 83 - DNA, 43 bp, Artificial Sequence
  Synthetic Oligonucleotide
gatggtctcggatccttactttttcttttttgcctggccggcc

SEQ ID NO: 84 - DNA, 31 bp, Artificial Sequence
  Synthetic Oligonucleotide
gctgaagatctcttgcagatagcagatccgg

SEQ ID NO: 85 - DNA, 31 bp, Artificial Sequence
  Synthetic Oligonucleotide
gaacgagctatacaaggaaacgacgctaggg

SEQ ID NO: 86 - DNA, 24 bp, Artificial Sequence
  Synthetic Oligonucleotide
gattgacagggtaatagagataaa

SEQ ID NO: 87 - DNA, 23 bp, Artificial Sequence
  Synthetic Oligonucleotide
aaactttatctctattaccctgt

SEQ ID NO: 88 - DNA, 24 bp, Artificial Sequence
  Synthetic Oligonucleotide
gattggggtaatagagataaaggg

SEQ ID NO: 89 - DNA, 23 bp, Artificial Sequence
  Synthetic Oligonucleotide
aaacccctttatctctattaccc

SEQ ID NO: 90 - DNA, 21 bp, Artificial Sequence
  Synthetic Oligonucleotide
caagttgataacggactagcc

SEQ ID NO: 91 - DNA, 23 bp, Artificial Sequence
  Synthetic Oligonucleotide
cttgctatttctagctctaaaac

SEQ ID NO: 92 - DNA, 21 bp, Artificial Sequence
  Synthetic Oligonucleotide
ttcccgacctgcaccaagcga

SEQ ID NO: 93 - DNA, 33 bp, Artificial Sequence; identical to SEQ ID NO: 48
  Synthetic Oligonucleotide

SEQ ID NO: 94 - DNA, 22 bp, Artificial Sequence
  Synthetic Oligonucleotide
gatggggtgccgctcgtgcttc

SEQ ID NO: 95 - DNA, 28 bp, Artificial Sequence
  Synthetic Oligonucleotide
agtccatggcaggtccagggttctcctc

SEQ ID NO: 96 - DNA, 20 bp, Artificial Sequence
  Synthetic Oligonucleotide
tggcatcayactttctacaa

SEQ ID NO: 97 - DNA, 20 bp, Artificial Sequence
  Synthetic Oligonucleotide
ccaccactdagcacaatgtt

SEQ ID NO: 98 - DNA, 69 bp, Artificial Sequence
  Synthetic Oligonucleotide
ggaagcggagctactaacttcagcctgctgaagcaggctggtgacgtggaggagaaccctggacctgcc

SEQ ID NO: 99 - DNA, 516 bp, Tomato bushy stunt virus
atggaacgagctatacaaggaaacgacgctagggaacaagctaacagtgaacgttgggatggaggatcaggaggaaccacttctcccttcaaacttcctgacgaaagtccgagttggactgagtggcggctacataacgatgagactaattcgaatcaagataatccccttggtttcaaggaaagctggggtttcgggaaagttgtatttaagagatatctcagatacgacaggacggaggcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactgaaaccttcgaaaaagaaagcgag

SEQ ID NO: 100 - protein, 172 aa, Tomato bushy stunt virus
MERAIQGNDAREQANSERWDGGSGGTTSPFKLPDESPSWTEWRLHNDETNSNQDNPLGFKESWGFGKVVFKRYLRYDRTEASLHRVLGSWTGDSVNYAASRFFGFDQIGCTYSIRFRGVSITVSGGSRTLQHLCEMAIRSKQELLQLAPIEVESNVSRGCPEGTETFEKESE

SEQ ID NO: 101 - protein, 22 aa, Artificial Sequence
  Synthetic Oligonucleotide
GSGATNFSLLKQAGDVEENPGP

SEQ ID NO: 102 - DNA, 3657 bp, Arabidopsis thaliana
aacacgaagtcacaaaaccctatttaaaagctttctactcttggacacagaatatctggtgctcagatttgtctctgcctattggacggtccagatttcaacattgtctctgatcctgtccctccattccaaatctttactttcaagttttaacatagaaagaaaaagaaaaaaaaaccttgtacttccttagtcctccttgttccatatctaactttatacttgtaagtttgatcaagaataatgttaattgttactatactaactaaacttaaaattaaacaatattgagcacattttcatatcttgtttattggttttggtgagattgagaattgaagaatctagagacaaaggcaaagctgtagtatagttgttgaatagggggtctacaactgtactgagtgaggggagggagagaatctcagttggagattgagagatctaaagtaatgataatagaaaatcttggaaaattaaattcttagaaaagaagaaagaaaaaataagatgagagagggaaagagagaggtggtccaaaaggaggtttaaggaagggtttgcaagtttgtatgaaaatagatagataaggaagatgatgtataaagccaaaactaacagttactatcttttcttctttttcttatattttcattttctcagaattcttttttacacttcccaaaaactcactcatttattcacctactctgcctccttttgtcctctatatagtctcttgtctacatttattttacttcacagtgtgcatatactatcaatttgtattagagagatctcaaagagaaatcaaaatggagtgataggttgtattatgaggattcgcagagcttttaaaatggcagccaacaacattctatgaactaagttaaacagtgttatgaaacataaataatttcatttgcaataaccaaagagaataatatttatttattttctttggctctatcatctaataggaggagaagtaaaaaaaacagaagagaaagatccaatcattctagaaaggaacacaatatgcttcacggatcccaagaatctttctatgcctgcctaaacccagcaatataaatcaaaccttcacacgcttcggttcttctttacacgtgccggaaaaaaaaccctagtagtagccgcccaatgaccatctaaagtggtccccgtgatgacacgtgtcagttggaccactatccgtaacttaacatgaaagcacatgtggggtcccctctttcgtcctttgccctaccagttccttgtcctagcccacaatacaatctacgcggtatctatatcaaagtttatctagctattttccgaaatagaaagcatatacttccatttatttttgaacaaattaaacttggtagaaataaaatctttcgatattgatttatttcgatttagtgtaattctattatcatctcgcgtgtcattctaggcttatagcaacagtgtaggtatgttgcaatgttgggttggtcatgccgtttggatttatttccagtgattaattcagattttatttttcttcttaattatctacgtataacaaaatctcgctaaccgcagagtgaatttgcatgtcactcatgaatgttttgagtataagaagtgagtaatttgttttataaatatatgaacttatgaagatacatattgaagttgttttgtttgggggtaaaaaaggttatttgagtgttatatgataactttactcagaaaacgtacttagcaaaggtaattcgaagtacctttggaatcgagtaaatactgataactagaaaaaataagatacataatggagaaataattaaatatatttgtatttcttttttgtttaacaacgtacgttttattattagctagtatacatttacaacggttacgtagatcatataatagccatttaagatgtacaacatctcatctggttacttcatttatataaaaaaaaaacgaaatctcaacacatagtaatgtataattacttcagtggggcttctcttaagacttgtattgagaatatccatataaaacaaactttgtattaagataattaaaattttctaatagtaggtattgggctgaagccaagattaacatggaggcagctttaaaatgtttccttatatgatgcagccatcatttctactctactccgtagctccaaacccttctcgtaattcacgtctctcatgctattctttttgctttcgtcctcctctcatgtgaagcaataactatctctcgattttttttttcaaataccgaaagctaactttttcaaataaatgtcaaatatattaattttcgttttgtatttagtattttatttgtcagctaagtatagtgagtttttaagcttactcgtcgtatttatcatatattcatatacatatcacattagtcaaagtaaataaaaatttgtttttgaagaaaaaaaaaatacatataactgcgagtctgcgactgtaactggacttgcttattttagttgatatgagctgagtaaaatcacgttgtcccagaccttgctcgctacaatcggcgaatggtctaacgtcccgacacctgtcctcgatccgcgggtactatattctttgcaatgtgatgcacgcgctgttactattggacagtgtttctcacctcacgactgagcctatgcgagtagcgacaatctccgatttgctgtctccatggtagggattatcacaatctctgattttttttatcaggaacaagtaaataaatagctttgagtttttgttttttttctacattcttcgcccaaaagatgtaagaaaataaaggatttgaaaccttgttctgttgttactcctttaaattcttaaaaactataaatcattatatctttgatctgtttcacaaactaatcatattcgttgcaaagtgagaattcgtcccactttactctttacaccgatactagtattatagatgtacagcatagtattccatatctagttatttagtcaaaactctatatattaagaggtaggttaattaattaaggagtaattgaagattatagaaagaataaaaaataccatttaatggacagaaccaaagataactaactatcatactataatgttgaatttcttccacgatccaatgcatggataacaacatcaatcaaatcatacattcatgctatataacatagttttcagttacaaactctcttttttatttatttcagttgttccttttcatgaccatattaacatcaaataatgcatttttttcaacgtctcttgacttacacccactaatattgacaaattgaacatctatacgactatacacacataagttaaaaatgcatgcaagtgctaagggaatttataacatctaaggttaataagactaagaaagtataaaataagaatacgtattatgaatttatgatatactttactaatctttttgaaaaatactttaatttaatctactatagggggtaaaaagtaaaaaagaaataaagatacgtttatccgcatatagtacctggaaataacagaaaataaaaacacaggtaagtactttgcctgagctagtatatgaacactaaagagatacacacacacaaaaagagagcagaaacaaaacaca

SEQ ID NO: 103 - DNA, 99 bp, Arabidopsis thaliana
gtttgtttgttttttaatcgttttcatcaacatgattgatatatatatagtttttgcacttgaaaaagttttgatttttatttatgtaaaaaactgcag

SEQ ID NO: 104 - DNA, 234 bp, Arabidopsis thaliana
aagaaacgtttggatggtgatcagaataatgtagttcgatccaacggtggtggattttcgaaatacacaatgattcctcctccgatgaacggctacgatcagtatcttcttcaatcagatcatcatcagaggagccaaggtttcctttatgatcatagaatcgctagagcagcttcagtttctgcttctagtactactattaatccttatttcaacgaggcaacaaatcatacg

SEQ ID NO: 105 - DNA, 90 bp, Arabidopsis thaliana
gtactaagtatagtccatttattaatactcatatataggtatatatgtatataactgttgatcttatttgatttaactggtgggtttagg

SEQ ID NO: 106 - DNA, 179 bp, Arabidopsis thaliana
gaccaatggaggaatttgggagctacatggaaggaaaccctagaaatggatcaggaggtgtgaaggagtacgagttttttccggggaaatatggtgaaagagtttcagtggtggctaaaacgtcgtcactcgtaggtgattgcagtcctaataccattgatttgtccttgaagctttaa

SEQ ID NO: 107 - DNA, 249 bp, Arabidopsis thaliana
atgttttatctttctatattgatttaaacaaaatcgtctctttaaagaaaaaacattttaagtagatgaaagtaagaaacagaagaaaaaaaagagagagccttttttggtgtatgcatctgagagctgagtcgaaagaaagattcagcttttggattacccttttggttgtttattatgagattctaacctaaacactcagacatatatgttctgttctcttccttaattgttgtcatgaaacttctc

SEQ ID NO: 108 - DNA, 1153 bp, Arabidopsis thaliana
tctccctctctctctctaaagttgtgtcagatcttggggcttgtatccattttgtgtttctttagttaggcaatgatcctgtcatgattcatgttatcacctaattaatcctcggttttcgaaacggtgtcatgcatatctgattaatcttgttatctggtgtgagataaatgtatcaaattgctcttgtttatagcttttccaattaatgtcattgcatagcatatgggatgtacagcttcgttttcgatttataaaatttatacatgtgtttggtacgtactgttttgttttggatcagaattataaatttagtacagtataaactactttaaaaattggccaatgagaattttgagtttatcttctttttataagttgattgattttgctttgtttatttatagaaaatatgaaattttatcaattatttttgggatgcttttttattgattttagttcggctaaaattagttagatgtttaaatttattaaaacagatatgcaaaaattaactcgatggtttttttttttttttttagggggataaaaatcgaactatatagttaattaaaacttaaaatgtttttctgaacggctatttcactgattttttagtagtttaaatgtttttcaccgaaaataaaaacagaataggaaacaatactatagaagtctaaccaaaaaaagttcaatatccagtagttcaatatttctaaaaagaaaatttctacacaaagtttatggcaaaaacctaagttattatggagaaaaccaagttattgaaagttatagcgtactgtatgtacaatctttcatcgtgatttcatgtattatagataaaattattcattgtggatgtaatgcaaatagaaccatccgtaaatccatgtattacggaaaaattattcatcacaactcttatgtttgaaaatagttagattcggtcttagatcattattttaaaaatacaaataaacaaatacgatcgacaacaattgctcagcaaatttgacatactgataaagtcattgtttgacaaaacacatatgggccttaatatcaaaacacacggcccattataaaagtaaactgataagataaaacagttccctatgattcctcaataagcagctgctcagcacgttctcgt

(19) United States
(12) Patent Application Publication
(10) Pub. No.: US 2016/0264982 A1
(43) Pub. Date: Sep. 15, 2016

(54) METHOD FOR PLANT GENOME SITE-DIRECTED MODIFICATION

(71) Applicant: SHANGHAI INSTITUTES FOR BIOLOGICAL SCIENCES, CHINESE ACADEMY OF SCIENCES, Shanghai (CN)

(72) Inventors: Jiankang Zhu, Shanghai (CN); Yanfei Mao, Shanghai (CN); Zhengyan Feng, Shanghai (CN); Botao Zhang, Shanghai (CN)

(21) Appl. No.: 14/905,070

(22) PCT Filed: Jul. 14, 2014
(86) PCT No.: PCT/CN2014/082144

§ 371 Date: Jan. 14, 2016

(30) Foreign Application Priority Data
Jul. 16, 2013 (CN) 201310299527.4
Sep. 4, 2013 (CN) 201310398734.5

(51) Int. Cl. C12N 15/82

(52) U.S. Cl. CPC C12N 15/8213; C12N 2800/80

(57) ABSTRACT

Provided is a method for plant genome site-directed modification. Specifically, a method for plant genome site-directed modification introduced by RNA is provided. By utilizing nucleic acid construct with particular structure, site-directed modification may be performed at pre-determined site in plant genome with high efficiency. Useful for screening plant with improved traits efficiently.

### TECHNICAL FIELD

The present invention relates to the field of biotechnology, in particular, to a RNA-guided targeted genome modification method for plants.

### BACKGROUND

Over the past decade, discovery and improvement of sequence-specific nuclease have exerted strong influence on the establishment of targeted mutagenesis. Zinc finger nuclease (ZFN) and Transcription activator-like effector nuclease (TALEN) are the main representatives (Carroll et al, 2006; Christian et al, 2010.). They are fusion proteins consisting of an engineered binding domain array for recognizing specific nucleic acid sequences and a non-specific nuclease Fok1 for DNA cleavage. The resulted double-strand breaks can be repaired via either the non-homologous end joining or the homologous recombination pathway in eukaryotic cells, thereby introducing site-specific nucleotides alteration or modification. The above mentioned techniques have been successfully applied in a number of species, including nematodes, human cells, mice, zebra fish, corn, rice, short grass, etc. (Beumer et al, 2006; Meng et al, 2008; Shukla et al., 2009; Meyer et al, 2010; Cui et al, 2011; Mahfouz et al, 2011; Li et al, 2012; Meyer et al, 2012; Shan et al, 2013; Weinthal et al, 2013). However, the main drawbacks of these techniques include low DNA recognition efficiency by protein elements, difficulty in engineering and vector construction and limitation of DNA recognition specificity.

In 2012, a breakthrough new technology was discovered and improved, CRISPR/Cas. CRISPR (clustered regulatory interspaced short palindromic repeats) is composed of short direct repeats separated by unique sequences of similar length. Functional CRISPR RNAs (crRNAs) are processed from transcripts of CRISPR array through base-pairing with another trans-activating crRNA (tracrRNA) at the direct repeats to form an RNA duplex that can be incorporated into Cas protein. And then, the binary complex will survey the genome for complementary DNA sequences and trigger double-strand breaks at the target sites.

Moreover, crRNA can be fused with tracrRNA to form a single-stranded chimeric RNA (chiRNA) molecule, which can also mediate the cleavage of targeted DNA sequences by Cas9 (Jinek et al., 2012). This editable type CRISPR/Cas system quickly achieved success applications in a number of species, including human cell lines, zebra fish, E. coli, mice and the like (Jinek et al, 2012; Hwang et al, 2013; Jiang et al, 2013; Jinek et al, 2013; Mali et al, 2013; Shen et al, 2013; Wang et al, 2013.). The main advantages of this technique include simplicity in vector construction, simultaneous gene-modifications at multiple target-sites. For animals, in vitro transcripts from chiRNA and Cas9 can be directly introduced (e.g. by injection) in embryonic cells, thereby causing heritable gene mutations. In mice, it was reported that genetic mutations have been successfully conducted to up to five target sites simultaneously. However, due to the presence of cell wall, such technique is not easy to apply in plants.

Summing up, to meet requirements on plant genetic engineering, there is an urgent need to develop a simple and efficient targeted gene modification method for plants.

### SUMMARY OF THE INVENTION

The object of the present invention is to provide a simple and efficient targeted gene modification method for plants.

Another object of the present invention is to provide a CRISPR/Cas toolkits suitable for plants to achieve successful and stable modification of targeted DNA sequences in progeny.

In the first aspect of the present invention, a targeted gene modification method for plant genome is provided, comprising the steps of:

(a) introducing a nucleic acid construct expressing chimeric RNA and Cas protein into a plant cell to obtain a transformed plant cell, wherein the chimeric RNA is a chimera consisting of CRISPR RNA (crRNA) specifically recognizing targeted sites to be modified (or to be cut) and trans-activating crRNA (tracrRNA); and

(b) under suitable conditions, forming chimeric RNA (chiRNA) through transcription of said nucleic acid construct in the transformed plant cell and expressing said Cas protein in said transformed plant cell, so that, in said transformed plant cell, site specific cleavage on genomic DNA is conducted by Cas protein under the guidance of said chimeric RNA, thereby performing targeted modification in genome.

In another preferred embodiment, said targeted modification includes random targeted modification and non-random targeted modification (precise targeted modification).

In another preferred embodiment, before genome DNA is cleaved by the chimeric RNA and Cas protein, a donor DNA is introduced into the plant cell, thereby performing precise targeted modification on genome. Said donor DNA is a single-stranded or double-stranded DNA and comprises DNA sequence to be inserted or replaced, and the DNA sequence may be a single nucleotide, or plurality of nucleotides (including DNA fragments or encoding genes).

In another preferred embodiment, said nucleic acid construct comprises a first nucleic acid sub-construct and a second nucleic acid sub-construct, wherein the first nucleic acid sub-construct and a second nucleic acid sub-constructs are independent from each other, or integrated;

wherein the first nucleic acid sub-construct comprises from 5′ to 3′ the following elements:
a first plant promoter;
encoding sequence of the chimeric RNA operably linked to the first plant promoter, and the encoding sequence of the chimeric RNA is shown in formula I:

A-B (I)
wherein,
A is DNA sequence encoding CRISPR RNA (crRNAs);
B is DNA sequence encoding trans-activating crRNA (tracrRNA);
“-” represents a linkage bond or a linker sequence between A and B; wherein a complete RNA molecule is formed through transcription of the encoding sequence of the chimeric RNA, i.e., the chimeric RNA (chiRNA); and
a RNA transcription terminator;

the second nucleic acid sub-construct comprises from 5′ to 3′ the following elements:
a second plant promoter;
encoding sequence of Cas protein operably linked to the second plant promoter, and the Cas protein is a fusion protein with nuclear localization sequence (NLS sequence) at N-end, C-end or both ends; and
a plant transcription terminator.

In another preferred embodiment, there is one or more of the first nucleic acid sub-construct (for multiple sites to be cut), and is independent to the second nucleic acid sub-construct, or the first nucleic acid sub-construct and the second nucleic acid sub-construct are integrated.

In another preferred embodiment, relative position between each of the first nucleic acid sub-construct and the second nucleic acid sub-construct is arbitrary.

In another preferred embodiment, the followings are operably linked from 5′ to 3′ between the second plant promoter and the encoding sequence of Cas protein:

the third nucleic acid sub-construct, and preferably, said third nucleic acid sub-construct is encoding sequence of p19 protein derived from Tomato bushy stunt virus (TBSV); and

self-splicing sequence, and preferably, said self-splicing sequence is encoding sequence of 2A polypeptide (SEQ ID NO.: 98).

In another preferred embodiment, the encoding sequence of p19 protein comprises the full-length sequence or cDNA sequence of p19 gene.

In another preferred embodiment, the sequence of 2A polypeptide is shown in SEQ ID NO.: 99.

In another preferred embodiment, the encoding sequence of p19 protein is shown in SEQ ID NO.: 100.

In another preferred embodiment, the amino acid sequence of p19 protein is shown in SEQ ID NO.: 101.

In another preferred embodiment, the targeted modifications include:

(i) in the absence of donor DNA, performing random insertions and deletions in specific sites of the plant genome; and

(ii) in the presence of donor DNA, performing precise insertion, deletion or replacement of DNA sequence in specific sites of the plant genome using the donor DNA as a template;

preferably, the targeted modification include gene knock-out, gene knock-in (transgene) of the plant genome and regulation (up-regulation or down-regulation) of the expression level of endogenous genes.

In another preferred embodiment, said RNA transcription terminator is U6 transcription terminator, which is at least 7 consecutive Ts (TTTTTTT).

In another preferred embodiment, the first plant promoter is an endogenous promoter from a plant to be modified.

In another preferred embodiment, the first plant promoter is RNA polymerase III-dependent promoter from a plant to be modified.

In another preferred embodiment, the RNA polymerase III-dependent promoter includes AtU6-26, OsU6-2, AtU6-1, AtU3-B, At7SL or combinations thereof.

In another preferred embodiment, the plant transcriptional terminator is Nos.

In another preferred embodiment, the second plant promoter is RNA polymerase II-dependent promoter, and preferably, comprises a constitutively expressed promoter or sporocyteless (SPL) promoter specifically expressed in Arabidopsis germline cell.

In another preferred embodiment, in the second nucleic acid sub-construct, expression cassette of SPL gene is, from 5′ to 3′, operably linked behind the encoding sequence of Cas protein.

In another preferred embodiment, the expression cassette of SPL gene comprises intron exon, untranslated region and terminator of SPL gene.

In another preferred embodiment, from 5′ to 3′, one or more sequences selected from the following group are operably linked to the expression cassette of SPL gene: sequence of SEQ ID NO.: 103 (intron 1), 104 (exon 2), 105 (intron 2), 106 (exon 3), 107 (3′ untranslated region), 108 (terminator).

In another preferred embodiment, the sequence of the plant transcription terminator in the second nucleic acid sub-constructs is shown in SEQ ID NO.: 108.

In another preferred embodiment, the nucleic acid construct is a plasmid simultaneously expressing the chimeric RNA and Cas protein.

In another preferred embodiment, the plant includes monocots, dicots and gymnosperms;

Preferably, said plant includes forestry plants, agricultural plants, crops, ornamental plants.

In another preferred embodiment, the plants include plants of the following families: Brassicaceae, Gramineae.

In another preferred embodiment, the plant includes but not limited to Arabidopsis, rice, wheat, barley, corn, sorghum, oats, rye, sugarcane, rapeseed, cabbage, cotton, soybean, alfalfa, tobacco, tomato, peppers, squash, watermelon, cucumber, apple, peach, plum, crabapple, sugar beet, sunflower, lettuce, lettuce, Artemisia annua, artichoke, stevia, poplar, willow, eucalyptus, clove, rubber trees, cassava, castor, peanut, peas, astragalus, tobacco, tomato and pepper.

In another preferred embodiment, said cas protein includes cas9 protein.

In another preferred embodiment, the second plant promoter is RNA polymerase II-dependent promoter.

In another preferred embodiment, RNA polymerase II-dependent promoter includes constitutive promoter and sporocyteless (SPL) promoter specifically expressed in Arabidopsis germline cell.

In another preferred embodiment, the first plant promoter includes AtU6-26, OsU6-2, AtU6-1, AtU3-B, At7SL or combinations thereof.

In another preferred embodiment, the second plant promoter includes 35s, UBQ, SPL promoter, or combinations thereof.

In another preferred embodiment, the method further comprises: before or after step (b), said transformed plant cell is regenerated into a plant.

In another preferred embodiment, the method further comprises: said transformed plant cell is detected for mutation or modification in genome.

In another preferred embodiment, the plant cell includes a plant cell derived from cultures, callus or plants.

In the second aspect, a nucleic acid construct used in targeted modification on plant genome is provided in the present invention, the nucleic acid construct comprising a first nucleic acid sub-construct and a second nucleic acid sub-construct, wherein the first nucleic acid sub-construct and the second nucleic acid sub-constructs are independent from each other, or integrated;

wherein the first nucleic acid sub-construct comprises from 5′ to 3′ the following elements:
the first plant promoter;
encoding sequence of the chimeric RNA operably linked to the first plant promoter, and the encoding sequence of the chimeric RNA is shown in formula I:

A-B (I)
wherein,
A is DNA sequence encoding CRISPR RNA (crRNAs);
B is DNA sequence encoding trans-activating crRNA (tracrRNA);
“-” represents a linkage bond or a linker sequence between A and B; wherein a complete RNA molecule is formed through transcription of the encoding sequence of the chimeric RNA, i.e., the chimeric RNA (chiRNA); and
a RNA transcription terminator;

the second nucleic acid sub-construct comprises from 5′ to 3′ the following elements:
a second plant promoter;
encoding sequence of Cas protein operably linked to the second plant promoter, and the Cas protein is a fusion protein with nuclear localization sequence (NLS sequence) at N-end, C-end or both ends; and
a plant transcription terminator.

In another preferred embodiment, the followings are operably linked from 5′ to 3′ between the second plant promoter and the encoding sequence of Cas protein:

the third nucleic acid sub-construct, and preferably, said third nucleic acid sub-construct is encoding sequence of p19 protein derived from Tomato bushy stunt virus (TBSV); and

2A sequence.

In another preferred embodiment, the encoding sequence of p19 protein comprises the full-length sequence or cDNA sequence of p19 gene.

In another preferred embodiment, the encoding sequence of p19 protein is shown in SEQ ID NO.: 98.

In another preferred embodiment, said RNA transcription terminator is U6 transcription terminator, which is at least 7 consecutive Ts (TTTTTTT).

In another preferred embodiment, the plant transcriptional terminator is Nos.

In another preferred embodiment, the nucleic acid construct is DNA construct.

In another preferred embodiment, the first nucleic acid sub-construct and the second nucleic acid sub-construct are integrated.

In another preferred embodiment, there is one or more of the first nucleic acid sub-construct (for multiple sites to be cut).

In another preferred embodiment, the first nucleic acid sub-construct and the second nucleic acid sub-construct is in the same plasmid.

In another preferred embodiment, the first nucleic acid sub-construct is located upstream or downstream to the second nucleic acid sub-construct.

In another preferred embodiment, the first plant promoter and/or second plant promoter is a constitutive or inducible promoter.

In another preferred embodiment, the encoding sequence of Cas protein further comprises NLS sequence located at both sides of ORF.

In another preferred embodiment, the second nucleic acid sub-construct further comprises Nos terminator located downstream to the encoding sequence of Cas protein.

In another preferred embodiment, the Cas protein further comprises a tag sequence.

In another preferred embodiment, the second nucleic acid sub-construct further comprises: a tag sequence (e.g. 3×Flag sequence) located between the second plant promoter and the encoding sequence of Cas protein.

In another preferred embodiment, the NLS sequence at N-end is located downstream to the tag sequence.

In the third aspect, a vector is provided in the present invention, said vector containing the nucleic acid construct according to the second aspect of the present invention.

The present invention also provides a vector combination, wherein the vector combination comprises a first vector and a second vector, wherein the first vector contains the first nucleic acid sub-construct of the nucleic acid construct according to the second aspect of the present invention, and the second vector contains the second nucleic acid sub-construct of the nucleic acid construct according to the second aspect of the present invention.

In another preferred embodiment, there is one or more of the first nucleic acid sub-construct.

In another preferred embodiment, there can be one or more of the first vector containing one or more of the first nucleic acid sub-construct of the nucleic acid construct according to the second aspect of the present invention.

In the fourth aspect, a genetically engineered cell is provided in the present invention, the cell containing the vector or vector combination according to the third aspect of the present invention.

In the fifth aspect, a plant cell is provided in the present invention, wherein the nucleic acid construct according to the second aspect of the present invention is integrated into the genome of said plant cell.

In the sixth aspect, a method for producing a plant is provided in the present invention, comprising the step of regenerating the plant cell according to the fifth aspect of the present invention into a plant.

In the seventh aspect, a plant is provided in the present invention, wherein the nucleic acid construct according to the second aspect of the present invention is integrated into the genome of plant cells in said plant.

In the eighth aspect, a plant is provided in the present invention, wherein the plant is prepared according to the method of the sixth aspect.

It should be understood that in the present invention, the technical features specifically mentioned above and below (such as in the Examples) can be combined with each other, thereby constituting a new or preferred technical solution which needs not be individually described.

### DESCRIPTION OF DRAWINGS

FIG. 1 shows that site-specific DNA double-strand break in Arabidopsis protoplasts can be caused by SpCas9 derived from Streptococcus pyogenes SF370. (A) Expression of SpCas9 is driven by 2×35S promoter, and guide RNA (chiRNA) is driven by AtU6-26 promoter in Arabidopsis. NLS, nuclear localization sequence; Flag, Flag tag sequence; Nos, Nos terminator. (B) YF-FP reporting system based on homologous recombination. In the figure, the designed target site of chiRNA is shown. PAM sequence is marked as purple, and the 20 bp target sequence is marked as blue-green. (C) CRISPR/Cas activity detected by YF-FP reporting system. YFP-positive cells are detected by flow cytometer.

FIG. 2 is a schematic diagram showing the stable transformation vector and designed sites in the target gene chiRNA. (A) The binary vector used in the Agrobacterium-mediated stable transformation of rice and Arabidopsis, which simultaneously contains chiRNA and Cas9 expression cassette. Expression of SpCas9 is driven by 2×35S promoter, Arabidopsis chiRNA is driven by AtU6-26 promoter, rice chiRNA is driven by OsU6-2 promoter. (B) Schematic diagram of target sites in Cas9/chiRNA. PAM sequence is marked as purple, chiRNA target site is marked as blue-green. Endonuclease sites are marked in frames. Restriction sites detected by RFLP are marked in black frames.

FIG. 3 shows that site-specific cleavage of DNA can be achieved by SpCas9 on multiple gene loci in Arabidopsis and rice strain. (A) and (B) Representative transgenic plant of T1 generation for targeting BRI1 locus 1. Plants which normally grow are shown in the left panels, and plants displaying similar phenotypes to bri1 mutants are shown in the right panels. The plants are screened on MS medium supplied with corresponding antibiotic for 5 days, transplanted into culture soil and cultured for one week (A) or three weeks (B), and then photographed. (C) Representative transgenic plant of T1 generation for GAI-bit locus 1. Plants which normally grow are shown in the left panels, and plants displaying similar phenotypes to gai mutants are shown in the right panels. The plants are screened on MS medium supplied with corresponding antibiotic for 5 days, transplanted into culture soil and cultured for four weeks, and then photographed. (D) Representative transgenic plant of T1 generation for targeting ROC5 locus 1 during rooting period. (E) Restriction analysis on 12 transgenic plants of T1 generation for BRI1 locus 1. PCR products are digested by EcoRV. M, DNA molecular weight standard. (F) Restriction analysis on 14 transgenic plants of T1 generation for ROC5 locus 1. PCR products are digested by AhdI. M, DNA molecular weight standard. (G) and (H) Representative type of mutation in target site detected in 1 transgenic seedling of T1 generation for BRI1 locus 1 (G) and ROC5 locus 1 (H). Wild-type control sequence is shown in the top, PAM sequence is marked as purple, and the target site is marked as blue-green. Red lines indicate deleted bases, and red letters indicate inserted or mutated bases. Whole changes of the sequence are marked in the right, wherein + means insertion, and D means deletion. (I) Statistical data of phenotypes and mutations observed in transgenic seedlings of T1 generation of rice and Arabidopsis. Scale length is 1 cm (A, B, C, D).

FIG. 4 shows that targeted deletion-mutations are induced by engineered chiRNA: Cas9 in BRI1 genelocus 1 of Arabidopsis. Indicated types of mutation are determined by amplifying genomic DNAs from 12 independent transgenic plants of T1 generation, cloning into a vector and sequencing. The sequence of the wild-type control is shown in the top of the figure, PAM sequence is marked as purple, and the target loci are marked as blue-green. Red lines indicate deleted bases, and red letters indicate inserted or mutated bases. Whole changes of the sequence are marked in the right, wherein + means insertion, and D means deletion. Note: for some sequences, both insertion and deletion are present. 75 mutations are detected in 98 clones.

FIG. 5 shows that targeted deletion-mutations are induced by engineered chiRNA: Cas9 in BRI1 genelocus 2 of Arabidopsis. Indicated types of mutation are determined by amplifying genomic DNAs from 3 independent transgenic plants of T1 generation, cloning into a vector and sequencing. The wild-type sequence is shown in the top of the figure, PAM sequence is marked as purple, and the target loci are marked as blue-green. Red lines indicate deleted bases, and red letters indicate inserted or mutated bases. Whole changes of the sequence are marked in the right, wherein + means insertion, and D means deletion. The number of detected mutations is shown in parentheses. 28 mutations are detected in 71 clones.

FIG. 6 shows that targeted deletion-mutations are induced by engineered chiRNA: Cas9 in BRI1 genelocus 3 of Arabidopsis. Indicated types of mutation are determined by amplifying genomic DNAs from 4 independent transgenic plants of T1 generation, cloning into a vector and sequencing. The wild-type sequence is shown in the top of the figure, PAM sequence is marked as purple, and the target loci are marked as blue-green. Red lines indicate deleted bases, and red letters indicate inserted or mutated bases. Whole changes of the sequence are marked in the right, wherein + means insertion, and D means deletion. The number of detected mutations is shown in parentheses. 22 mutations are detected in 34 clones.

FIG. 7 shows that targeted deletion-mutations are induced by engineered chiRNA: Cas9 in GAI genelocus 1 of Arabidopsis. Indicated types of mutation are determined by amplifying genomic DNAs from 3 independent transgenic plants of T1 generation, cloning into a vector and sequencing. The wild-type sequence is shown in the top of the figure, PAM sequence is marked as purple, and the target loci are marked as blue-green. Red lines indicate deleted bases, and red letters indicate inserted or mutated bases. Whole changes of the sequence are marked in the right, wherein + means insertion, and D means deletion. The number of detected mutations is shown in parentheses. 17 mutations are detected in 53 clones.

FIG. 8 shows that targeted deletion-mutations are induced by engineered chiRNA: Cas9 in ROC5 gene locus 1 of Arabidopsis. Indicated types of mutation are determined by amplifying genomic DNAs from 5 independent transgenic plants of T1 generation, cloning into a vector and sequencing. The wild-type sequence is shown in the top of the figure, PAM sequence is marked as purple, and the target loci are marked as blue-green. Red lines indicate deleted bases, and red letters indicate inserted or mutated bases. Whole changes of the sequence are marked in the right, wherein + means insertion, and D means deletion. The number of detected mutations is shown in parentheses. 136 mutations are detected in 165 clones.

FIG. 9 shows the Pa7-YFP vector.

FIG. 10 shows the sequence of AtU6-26 chiRNA (target site recognition sequence SEQ ID NO.: 1 is not inserted). In the sequence, AtU6-26 promoter is marked as gray, two BbsI restriction sites in insertion target site oligo are underlined, and the area in trans-activating crRNA which will be fused with target site is marked in a frame.

FIG. 11 shows the sequence of AtU6-26 chiRNA (target site recognition sequence SEQ ID NO.: 2 is inserted).

FIG. 12 shows the sequence of OsU6-2 chiRNA (target site recognition sequence SEQ ID NO.: 3 is not inserted). In the sequence, OsU6-2 promoter is marked as gray, two BbsI restriction sites in insertion target site oligo are underlined, and the area in trans-activating crRNA which will be fused with target site is marked in a frame.

FIG. 13 shows the sequence of OsU6-2 chiRNA (target site recognition sequence SEQ ID NO.: 4 is inserted).

FIG. 14 shows the sequence of 2×35S-Cas9-Nos (SEQ ID NO.: 39).

FIG. 15 shows that targeted mutations in both CHLI1 and CHLI2 genes in transgenic plants of T1 generation of Arabidopsis caused by CRISPR-Cas9. Indicated types of mutation are determined by amplifying genomic DNAs from 3 independent transgenic plants of T1 generation, cloning into a vector and sequencing. The sequence of wild-type control is shown in the top of the figure, and target sites are underlined. Whole changes of the sequence are marked in the right, wherein + means insertion, and − means deletion.

FIG. 16 shows that targeted mutations of two sites in TT4 gene in transgenic plants of T1 generation of Arabidopsis and large fragment deletion between the target sites are caused by CRISPR-Cas9. Indicated types of mutation are determined by amplifying genomic DNAs from 11 independent transgenic plants of T1 generation, cloning into a vector and sequencing. The sequence of wild-type control is shown in the top of the figure, and target sites are underlined. Whole changes of the sequences in two sites and detected ratio are marked in the right, wherein + means insertion, and − means deletion.

FIG. 17 shows a schematic diagram for constructing CRISPR/Cas9 vectors for plant genes targeting. pSPL-Cas9-sgR: CRISPR/Cas9 vector for plant gene targeting in germline cells. pUBQ-Cas9-sgR: constitutively expressed CRISPR/Cas9 vector for plant gene targeting. pAtU6: promoter of U6 gene in Arabidopsis; sgRNA: single-stranded guide RNA; pAtSPL: promoter of SPL gene in Arabidopsis; pAtUBQ: promoter of UBQ gene in Arabidopsis; HspCas9: humanized Cas9 gene in Streptomyces; SPL intron: intron of SPL gene; SPL exon: exon of SPL gene; tSPL: terminator of SPL gene; tUBQ: terminator of UBQ gene.

FIG. 18 shows in situ hybridization of Cas9 gene. A, B, C: T1 transgenic plants of pSPL-Cas9-sgR; D, E, F: T1 transgenic plants of pUBQ-Cas9-sgR. A, D: phase V of anther development; B, E: phase VII of anther development; C, F: phase II of ovule development. Scale=20 μM.

FIG. 19 shows the statistics of efficiency of plant gene targeting using the germline-specific system. A: based on the alignment of sequencing results, it is found that no mutation is detected in the transgenic plants of T1 generation for pSPL-Cas9-sgR-AP1-27/194, while in the corresponding plants of T2 generation, mutations can be detected. B: comparison of knockout efficiency between constitutive gene targeting system and germline cell-specific gene targeting system in different tissues and different generations.

FIG. 20 shows the statistics of mutation types in T2 transformants of different plant targeting systems. With respect to each T2 population transformed with different targeting constructs, 8 mutated strains are randomly selected, and 12 single plants are respectively detected for the statistics of mutation types.

FIG. 21 shows a schematic diagram of a highly efficient gene-targeting construct for plants. A: psgR-Cas9: a general gene-targeting construct for Arabidopsis. B: psgR-Cas9-p19: a modified gene targeting construct with the plant post-transcriptional gene silencing suppressor co-expressed. pAtU6: U6 gene promoter in Arabidopsis; sgRNA: single-stranded guide RNA; pUBQ: UBQ gene promoter in Arabidopsis; hSpCas9: humanized Cas9 gene in Streptomyces; tUBQ: terminator of UBQ gene in Arabidopsis; TBSV-p19: encoding gene of p19 protein from Tomato bushy stunt virus (TBSV); 2A peptide: protein cis-cutting element; BbsI: endonuclease recognition site of BbsI.

FIG. 22 shows the gene targeting efficiency of the p19 co-expressed construct detected by transient expression assay in protoplasts. A: schematic diagram showing the functional mechanism of p19 and the principle of signal detection. p19 protein is present in plant cells as a dimer, and can inhibit degradation of sgRNA and improve the binding activity of sgRNA with Cas9. sgRNA-Cas9 complex can bind to the recognition site on YFFP report-gene and trigger double-stranded DNA breaks (DSB) by cleavage. Certain partially duplicated YFP sequence will be subject to single-strand annealing, excised by DNA damage repair system and corrected. B: Fluorescence detection of YFFP transient expression system. a, c, e, g, I, k: signal from positive cells under YFP fluorescence channel. b, d, f, h, j, l: autofluorescence signal from chloroplast under RFP fluorescence channel. Values indicated at bottom left side represent the percentage of YFP-positive cells in the whole cell population.

FIG. 23 shows the gene expression analysis of sgR-Cas9-p19 transgenic plant. A: three leaf developmental phenotypes with different degrees are present in sgR-Cas9-p19 transgenic population: 1/−: flat leaves, 2/+: curl leaves, 3/++: serrated leaves. B: According to Northern Blotting results, it is showed that, in the transgenic plant with serrated leaves, the expression level of sgRNA and miR168 are significantly increased. C, D: According to Realtime PCR results, it is showed that there is a positive correlation between the degree of leaf developmental phenotype and the expression level of p19, however, the expression of Cas9 gene is relatively stable.

FIG. 24 shows phenotype analysis of sgR-Cas9-p19 transgenic plant of T1 generation. According to the severity of leaf developmental defects, transgenic plants of sgR-Cas9-p19-AP1 and sgR-Cas9-p19-TT4 can be classified into 3 types: no phenotype (p19/−), curl leaves (p19/+) and serrated leaves (p19/++). And according to the degree of targeted gene mutations, the transgenic plants can also be classified into 3 types: wild-type (WT), chimera and mutant. The number of corresponding plants is recorded respectively, and summarized in a table.

### MODE FOR CARRYING OUT THE INVENTION

Through comprehensive and intensive research, RNA-guided targeted genome modification in plants has been successfully achieved by the inventors by using nucleic acid constructs of specific structure. Using the method of the present invention, targeted cleavage and modification can be performed and a variety of different types of mutations can be efficiently introduced into specific sites, thereby facilitating the screening of modified new plants. And the proportion of genetically modified plants can be increased in transgenic offspring of the germline specific gene targeting system. Moreover, the inventors have also discovered that when a specific sequence is introduced into the nucleic acid construct of the present invention, the targeting efficiency in plants can be effectively improved and the developmental phenotype of a plant can be influenced. Based on the above findings, the present invention is completed.

Based on the experimental results, the present invention is particularly applicable to plants, and targeted cleavage on DNA sequence and gene modification in genome can be achieved in a stably inherited plant.

### DEFINITION

As used herein, the term “crRNA” refers to CRISPR RNA which is responsible for recognizing target sites.

As used herein, the term “tracrRNA” refers trans-activating crRNA pairing with crRNA.

As used herein, the term “plant promoter” refers to a nucleic acid sequence initiating transcription of nucleic acid in a plant cell. The plant promoter may be derived from plants, microorganisms (such as bacteria, viruses) or animals, or an artificially synthesized or engineered promoter.

As used herein, the term “plant transcription terminator” refers to a terminator which can terminate transcription in plant cells. The plant transcription terminator may be derived from plants, microorganisms (such as bacteria, viruses) or animals, or an artificially synthesized or engineered terminator. Representative examples include (but are not limited to): Nos terminator.

As used herein, the term “Cas protein” refers to a nuclease. A preferred Cas proteins are Cas9 protein. Typical Cas9 protein includes (but not limited to): Cas9 derived from Streptococcus pyogenes SF370.

As used herein, the term “encoding sequence of Cas protein” means a nucleotide sequence encoding Cas protein with cleavage activity. In the case where the inserted polynucleotide sequence is transcribed and translated to produce functional Cas protein, a skilled person will appreciate that a large number of polynucleotide sequences can encode the same polypeptide due to codon degeneracy. In addition, a skilled person will also appreciate that different species will have certain preference for codon, and codons for Cas protein will be optimized according to requirements on expression in different species. These variants should be included into term “encoding sequence of Cas protein”. Furthermore, the term specifically includes full-length sequence of Cas gene sequence, a sequence which is substantially identical with Cas gene sequence, and a sequence encoding a protein which maintain the function of Cas protein.

As used herein, the term “plant” includes complete plants, plant organs (e.g., leaves, stems, roots, etc.), seeds and plant cells as well as progeny thereof. It is not necessary to particularly limit the type of plant which can be used in the method of the present invention, generally including any type of higher plants suitable for transformation, including monocots, dicots and gymnosperms.

As used herein, the term “heterologous sequence” is a sequence from different species, or, if from the same species, a sequence highly modified from its original form. For example, a heterologous structural gene operably linked to a promo er may be derived from a different species from which the structural gene is originally obtained, or, if from the same species, one or both of them are highly modified from their original forms.

As used herein, “operably linked to” or “operably linked” refers to a situation in which some parts of a linear DNA sequence can affect the activity of other parts in the same linear DNA sequence. For example, if a signal peptide DNA is expressed as a precursor and involves in the secretion of polypeptide, then the signal peptide (secretory leader sequence) DNA is operably linked to polypeptide DNA; if a promoter controls transcription of a sequence, then it is operably linked to encoding sequence; and if a ribosome binding site is positioned in a position where it can be translated, then it is operably linked to encoding sequence. Generally, “operably linked to” means “neighbor”, and, for secretion leader sequence, it means “neighbor” in reading frame.

As used herein, the term “encoding sequence of 2A polypeptide”, “self-splicing sequence”, or “2A sequence” refers to a protease-independent self-splicing amino acid sequence found in virus, similar to IRES. Using 2A, simultaneous expression of two genes from a single promoter can be achieved. It is also widely found in various types of eukaryotic cells. Unlike IRES, the expression level of downstream proteins will not be reduced. However, after splicing, residues of 2A polypeptide will linked to the upstream protein as a single entity, and Furin proteolytic cleavage site (4 basic amino acid residues, such as Arg-Lys-Arg-Arg) can be added between the upstream protein and 2A polypeptide to completely remove the residues of 2A polypeptide from the end of upstream protein.

As used herein, the term “chimeric RNA (chiRNA)”, “single-stranded guide RNA (sgRNA)” are used interchangeably to refer to a RNA sequence, which contains encoding sequence of the structure of formula I and is capable of forming a complete RNA molecule through transcription.

Nucleic Acid Construct

A nucleic acid construct is provided in the present invention, said nucleic acid construct comprising a first nucleic acid sub-construct and a second nucleic acid sub-construct, wherein the first nucleic acid sub-construct and the second nucleic acid sub-construct are independent from each other, or integrated;

wherein the first nucleic acid sub-construct comprises from 5′ to 3′ the following elements:

a first plant promoter;

encoding sequence of the chimeric RNA operably linked to the first plant promoter, and the encoding sequence of the chimeric RNA is shown in formula I:

A-B (I)

wherein,

A is DNA sequence encoding CRISPR RNA (crRNAs);

B is DNA sequence encoding trans-activating crRNA (tracrRNA);

“-” represents a linkage bond or a linker sequence between A and B; wherein a complete RNA molecule is formed through transcription of the encoding sequence of the chimeric RNA, i.e., the chimeric RNA (chiRNA); and

a RNA transcription terminator (including but not limited to: U6 transcription terminator, at least 7 consecutive Ts);

the second nucleic acid sub-construct comprises from 5′ to 3′ the following elements:

a second plant promoter;

encoding sequence of Cas protein operably linked to the second plant promoter, and the Cas protein is a fusion protein with nuclear localization sequence (NLS sequence) at N-end, C-end or both ends;

a plant transcription terminator (including but not limited to Nos terminator, etc.).

In the present invention, the strength of the first plant promoter and the second plant promoter can initiate production of an effective amount of chiRNA and Cas protein, for achieving site-directed modification for plant genome.

In the present invention, it should be understood that the first nucleic acid sub-construct and the second nucleic acid sub-construct may be located on the same polynucleotide or different polynucleotides, or can also be located on the same vector or different vectors.

The above mentioned nucleic acid construct constructed in the present invention can be introduced into plant cells by conventional recombinant techniques for plant (e.g. Agrobacterium transfection technique), thereby obtaining plant cells containing the nucleic acid construct (or a vector containing the nucleic acid construct), or obtaining plant cells with said nucleic acid construct integrated into the genome.

In the plant cell, chiRNA formed through transcription of the nucleic acid construct of the present invention pairs with the expressed Cas protein, to site-specifically cleave genome, thereby introducing a variety of different mutations.

Furthermore, in order to obtain more seeds containing mutated genes, further improve the activity of CRISPR/Cas9 system in germline cells and reduce possible adverse effects on plant development from gene targeting technique, expression cassette of Arabidopsis SPOROCYTELESS (SPL) gene is used in the present invention to drive expression of Cas9 genes.

SPL gene is specifically expressed in germline cells of Arabidopsis, including megasporocyte and microsporocyte. According to in situ hybridization experiment, it is demonstrated that transcription of Cas9 can be effectively initiated in germline cells by using expression cassette of SPL gene. And the results of mutant detection also demonstrate that Cas9 expression system driven by SPL promoter won't affect the gene function, growth and development of T1 transgenic plants. However, a great deal of heterozygotes, in which targeted genes are mutated, can be obtained in the transgenic population of T2 generation, indicating that the mutation of target gene occurs in germline cells.

For further improving the stability of sgRNA in plants and efficiency of gene-targeting of CRISPR/Cas9 system, a gene-targeting vector psgR-Cas9-p19, co-expressing TBSV-p19 protein and Cas9 protein is constructed. The protein activity of the correctly recombined YFFP gene is detected in Arabidopsis transient expression system, and based on the results, it was showed that p19 protein can significantly improve the gene-targeting efficiency of CRISPR/Cas9 system.

Furthermore, p19 co-expression vector targeting Arabidopsis endogenous genes is constructed, and clear leaf developmental phenotypes can be found in about one-third of the obtained plants of T1 generation suggesting that p19 will inhibit the miRNA-regulated development process in plants. Results from Northern detection and quantitative analysis on gene expression show that the expression level of p19 protein is positively correlated with the cumulative amount of miR168 and sgRNA. Meanwhile, analysis on phenotype and genotype of target sites also shows that the higher the expression of p19 in transgenic plants, the higher the probability of mutation in a target gene, which provides important basis and means for further improving plant gene-targeting system based on CRISPR/Cas9.

Method for Targeted Gene Cleavage

A method for targeted gene cleavage or modification on the genome of plants is also provided in the present invention.

(a) a nucleic acid construct expressing chimeric RNA and expressing Cas protein is introduced into a plant cell to obtain a transformed plant cell; and

(b) under suitable conditions, the nucleic acid construct in the transformed plant cell is transcribed to form chimeric RNA (chiRNA), and the transformed plant cell expresses said Cas protein, so that targeted cleavage on genome is performed by said Cas protein in said transformed plant cell, under the guidance of the chimeric RNA, thereby performing targeted modification on genome.

In the method of the present invention, in step (a), the nucleic acid constructs expressing chimeric RNA and expressing Cas protein can be in the same nucleic acid construct, or may be in different nucleic acid constructs.

In addition, if Cas protein expression cassette has been contained in the plant or plant cell to be treated, merely a nucleic acid construct expressing chimeric RNA can be introduced.

Further, if it is necessary to perform targeted cleavage or targeted modification at multiple specific sites, a nucleic acid construct expressing a plurality of different chiRNAs (may be in the same or in different nucleic acid constructs) may be introduced into a plant cell.

Upon targeted cleavage, plant cells will be repaired through a variety of mechanisms, and a variety of mutations may often be introduced during the repair process. Based on this, plants or plant cells with desired mutation and desired performance can be screened for use in subsequent research or production.

Method for Precise Targeted Genome Modification

If it is necessary to preform precise targeted insertion, deletion or replacement of DNA sequence in plant genome, a donor DNA can be introduced before the initiation of targeted gene cleavage on genome by chimeric RNA and Cas protein. The donor DNA can be a single-stranded or double-stranded DNA, and contain DNA sequence to be inserted and replaced. The DNA sequence may be a single nucleotide, or a plurality of nucleotides (including DNA fragment or encoding gene). Upon targeted cleavage, precise targeted insertion, deletion or replacement for plant genome can be performed in a plant cell through homologous recombination-mediated DNA repair system and using donor DNA as a template. The donor DNA can be inserted into a specific location in plant genome or used to replace specific DNA sequences; or can also be used to replace promoter, and insert enhancer or other DNA cis-regulatory elements to regulate the expression level of endogenous genes in a plant; and also be used to insert a polynucleotide sequence encoding a complete protein. The methods for introducing donor DNA include, but not limited to: microinjection, Agrobacterium-mediated transfection, gene-gun, electroporation, ultrasonic method, liposome-mediated method, polyethylene glycol (PEG) mediated method, laser microbeam puncture, direct-introduction of donor DNA after chemical modification (adding lipophilic groups) and the like.

Use

The present invention can be used in plant genetic engineering for modifying various plants, especially crops and forestry plants with economic value.

The main advantages of the present invention include:

(a) targeted cleavage and modification can be specifically performed at specific positions in a plant genome;

(b) various forms of modifications can be efficiently introduced into specific positions;

(c) new genes can be efficiently introduced into specific positions.

(d) specific genes in the plant genome can be efficiently knock out.

(e) expression level of endogenous genes in a plant can effectively regulated.

The invention will be further illustrated with reference to the following specific examples. It is to be understood that these examples are only intended to illustrate the invention, but not to limit the scope of the invention. For the experimental methods in the following examples without particular conditions, they are performed under routine conditions, such as conditions described in Sambrook et al., Molecular Cloning: A Laboratory Manual, New York: Cold Spring Harbor Laboratory Press, 1989, or as instructed by the manufacturer. All the percentages or fractions refer to weight percentage and weight fraction, unless stated otherwise.

General Materials and Methods

Growth of Arabidopsis and Rice

Wild-type Arabidopsis Col-0 (available from the American ABRC center) is used in experiments. Seeds are inoculated on MS medium and vernalized at 4° C. for 3 days, and then placed into long photoperiod growth chamber (16 h light/8 h darkness) at 22° C., and after 5-10 days, the seedlings are transplanted to nutrient soil.

Rice used in the experiment is Kasalath cultivar (purchased from China Rice Research Institute). After transplanted to soil, the plants are grown in a greenhouse (16 h light, 30° C./8 h darkness, 22° C.).

Design of Target Sites

Suitable target sites for chiRNA is in the form of N₁₋₂₀NGG, wherein N₁₋₂₀ is recognition sequence provided by chiRNA vector construct, and NGG is a recognition sequence necessary for CRISPR/Cas9 complex binding to DNA target sites, called PAM sequence.

G is used as starting signal for transcription of U6 type small RNA, therefore, sequence in the form of GN₁₉NGG is selected as target sites. In addition, according to previous study, it was shown that CRISPR/Cas system can tolerate mismatch of target site from the side of PAM sequence up to five bases, therefore, if the first nucleotide in N₁₋₂₀ is G, the synthesized oligo primer for target site is linker+N₁₋₂₀; and if the first nucleotide in N₁₋₂₀ is not G, it will be deemed as G in the present Examples, and the synthesized oligo primer for target site will be linker+GN₂₋₂₀.

Construction of Vector

Encoding sequence of SpCas9 was PCR-amplified from vector PX260 by using primers Cas9-F and Cas9-R, and subcloned between the XhoI and BamHI sites of pA7-GFP vector to replace its original GFP gene, thereby obtaining 2×35S promoter and Nos terminator at N-terminal and C-terminal respectively. Detailed construction of pX260 and A7-GFP vector can be found in literature (Voelker et al., 2006; Cong et al., 2013). Afterwards, complete expression cassette from 2×35S promoter to Nos terminator is subcloned into pBluescript SK+vector (commercially available from Stratagene Inc., San Diego, Calif.) by HindIII/EcoRI restriction sites, named as 35S-Cas9-SK.

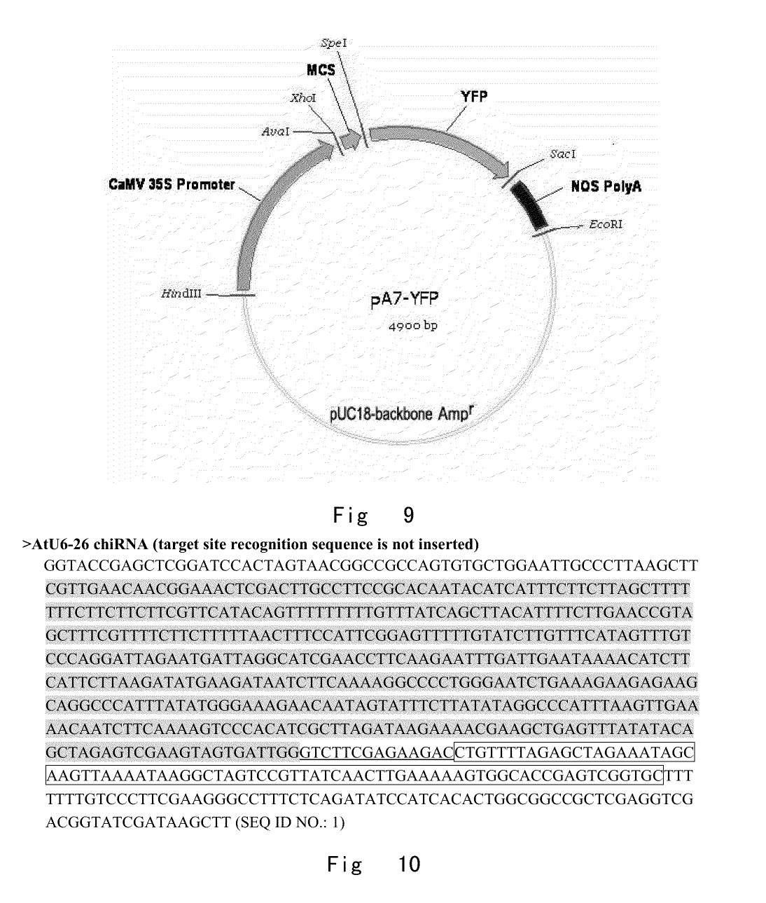

AtU6-26 promoter is obtained through PCR amplification using AtU6-26F and AtU6-26R as primers and wild-type Arabidopsis thaliana Col-0 genome DNA as a template, and subcloned into pEasy-Blunt vector (available from TransGen Biotech, Beijing), and a clone with KpnI preceding the promoter is selected. Afterwards, it is subcloned into pBluescript SK+vector (purchased from Stratagene Inc., San Diego, Calif.) using KpnI/XhoI restriction site. 85 bp of chiRNA inducing sequence is obtained through PCR amplification from pX330 vector using Atu6-26-85F and AtU6-26-85R primers and fused with AtU6-26 promoter to obtain a complete chiRNA expression vector (see FIG. 10), and the obtained vector was named as At6-26SK. Upstream and downstream oligonucleotide strands (see Table 1) are synthesized according to the designed target sites, and double-stranded small fragment with linkers formed by annealing is cloned between two BbsI sites of BbsI-digested At6-26SK through ligation reaction.

chiRNA expression cassette is subcloned into 35S-Cas9-SK through KpnI/EcoRI digestion for transient expression analysis; or digested using KpnI/SalI, and then subcloned into KpnI/EcoRI region of pCambia1300 vector (Cambia, Canberra, Australia) along with SalI/EcoRI fragment containing complete Cas9 expression cassette for transgene of Arabidopsis.

OsU6-2 promoter is obtained through PCR amplification using OsU6-2F and OsU6-2R as primers and Wild type rice Nipponbare genome DNA as template, and then subcloned into pEasy-Blunt vector (TransGen Biotech, Beijing).

OsU6-2 is transferred into At6-26SK vector to replace AtU6-26 promoter through Transfer PCR by using TPCR-OSu6F and TPCR-OsU6R primers method, thereby obtaining OsU6-2SK vector (see FIG. 12). Upstream and downstream oligonucleotide strands are synthesized according to the designed target sites, and double-stranded small fragment with linkers formed by annealing is cloned between two BbsI sites of BbsI-digested OsU6-2SK through ligation reaction. chiRNA expression cassette is subcloned into 35S-Cas9-SK through KpnI/EcoRI digestion for transient expression analysis; or digested using KpnI/HindIII, and then subcloned into KpnI/EcoRI region of pCambia1300 vector (Cambia, Canberra, Australia) along with HindIII/EcoRI fragment containing complete Cas9 expression cassette for transgene of rice.

pAtU6-26 fragment of AtU6-26 promoter is obtained through PCR amplification using pAtU6-F-HindIII and pAtU6-R as primers and wild-type Arabidopsis thaliana Col-0 genome as a template. chiRNA (i.e., SgRNA) fragment is obtained through PCR amplification by using sgR-F-U6 and sgR-R-SmaI primers and pX330 vector as a template. pAtU6-chiRNA fragment (SEQ ID NO.: 40) is obtained through overlapping PCR by using pAtU6-F-HindIII and sgR-R-SmaI primers and mixture of PCR products of chiRNA and pAtU6 as a template, digested by HindIII and XmaI and inserted into corresponding sites of pMD18T vector to give PSGR-At vector.

pAtUBQ1 promoter and terminator of AtUBQ1 are obtained through PCR amplification using pAtUBQ1-F-SmaI and pAtUBQ1-R-Cas as well as tUBQ1-F-BamHI and tUBQ-R-KpnI primers and wild-type Arabidopsis thaliana Col-0 genome as a template. Cas9 gene fragment is obtained through PCR amplification by using Cas9-F-pUBQ and Cas9-R-BamHI as primers and pX330 vector as a template. The above pAtUBQ1, Cas9 gene and terminator fragment of AtUBQ1 are digested with XmaI and NcoI, NcoI and BamHI, as well as BamHI and KpnI, and ligated into psgR-At vector digested with XmaI and KpnI, thereby finally obtaining psgR-Cas9-At backbone vector with pAtUBQ-Cas9-tUBQ (SEQ ID NO.: 41) as insert fragment.

Sequence complying with 5′-NNNNNNNNNNNNNNNNNNNNGG-3′ is selected as a target. For psgR-Cas9-At vector, sense strand 5′-GATTGNNNNNNNNNNNNNNNNNNN-3′ and antisense strand 5′-AAACNNNNNNNNNNNNNNNNNNNC-3′ were synthesized respectively. Then double-stranded DNA small fragment with linkers formed by denaturing and annealing both of the synthesized artificial sequences is inserted between two BbsI sites of psgR-Cas9-At, thereby obtaining psgR-Cas9-At vector for specific target sites. Complete pAtU6-chiRNA element is amplified from psgR-At vector with inserted target gene fragment by using pAtU6-F-KpnI and sgR-EcoRI as primers, digested with KpnI and EcoRI, and inserted into psgR-Cas9-At vector with pAtU6-chiRNA element for another target gene, thereby obtaining p2×sgR-Cas9-At vector. Afterwards, the vector is digested with HindIII and EcoRI, and complete 2×sgr-Cas9-At is subcloned into pCambia1300 vector (Cambia, Canberra, Australia) to obtain binary vector p2×1300-sgr-Cas9 for transgene of Arabidopsis.

Construction of pUBQ-Cas9-sgR Series Vectors

Primers sgR-Bsa I-F/R are synthesized, and the primers are added with phosphorus by PNK kinase, slowly anneal, and are linked into Bbs I site of psgR-Cas9-At. The resulting psgR-Cas9-Bsa vector is digested with EcoR I and HindIII and linked into pBin19 vector, thereby obtaining pUBQ-Cas9-sgR vector. Synthesized primers sgR-AP1-S27/A27 and sgR-AP1-S194/A194 are also linked into BsaI site of pUBQ-Cas9-sgR vector according to the above method, thereby obtaining pUBQ-Cas9-sgR-AP1-27 and pUBQ-Cas9-sgR-AP1-194.

Construction of pSPL-Cas9-sgR Series Vector

Primers SPL5′-F-XmaI and SPL5′-R-BsaI are synthesized, and promoter sequence at 5′end of SPL gene is amplified from Arabidopsis genome. This fragment is digested with Xma I and Bsa I, and linked into Xma I and Nco I sites of psgR-Cas9-Bsa, thereby obtaining pSPL-Cas9-5′. Primers SPL3′-F-BamHI and SPL3′-R-KpnI are synthesized, and promoter sequence at 3′end of SPL gene is amplified from Arabidopsis genome, which comprises exons (SEQ ID NO.: 104, 106), two introns (SEQ ID NO.: 103, 105) and terminator (SEQ ID NO.: 108) after SPL gene, digested with BamH I and Kpn I and linked into pSPL-Cas9-5′, to give PSPL-Cas9-53′. The resulting plasmid is digested with Xma I and Kpn I, and linked into pUBQ-Cas9-sgR, thereby obtaining pSPL-Cas9-sgR vector. The synthesized primers sgR-AP1-S27/A27 and sgR-AP1-S194/A194 are also linked into Bsa I site of pSPL-Cas9-sgR vector according to the above method, thereby obtaining pSPL-Cas9-sgR-AP1-27 and pSPL-Cas9-sgR-AP1-194.

Construction of psgR-Cas9-p19 Vector

TBSV-p19-2A gene containing Nco I site is synthesized by GENEWIZ, Inc. The gene fragment is digested with NcoI, and then inserted into NcoI site of psgR-Cas9 vector. The insertion direction of the fragment is identified by using p19-F and Cas9-378R primers, thereby obtaining psgR-Cas9-p19 vector.

Construction of psgR-Cas9-MRS1/2 Vectors

Primers sgR-MRS1-S/A and sgR-MRS2-S/A are synthesized respectively, and linked into Bbs I site of psgR-Cas9-At, thereby obtaining psgR-Cas9-MRS1 and psgR-Cas9-MRS2 vectors.

Construction psgR-Cas9-MRS1/2-p19 Vectors

Primers sgR-MRS1-S/A and sgR-MRS2-S/A are synthesized respectively, and linked into Bbs I site of psgR-Cas9-p19, thereby obtaining psgR-Cas9-MRS1-p19 and psgR-Cas9-MRS2-p19 vectors.

Construction of 1300-psgR-Cas9-p19-AP1/TT4 Vector

Primers sgR-AP1-S27/A27, sgR-AP1-S194/A194, sgR-TT4-S65/A65 and sgR-TT4-S296/A296 are synthesized respectively, and the primers are added with phosphorus by using PNK kinase, anneal, and are linked into Bbs I site of psgR-Cas9-p19, thereby obtaining psgR-Cas9-p19-AP1-27, psgR-Cas9-p19-AP1-194, psgR-Cas9-p19-TT4-65 and psgR-Cas9-p19-TT4-296. psgR-Cas9-AP1-194-p19 and psgR-Cas9-p19-TT4-296 are amplified by using AtU6-F-KpnI and sgR-R-EcoRI primers, and the resulting fragments are digested by using Kpn I and EcoR I and linked into psgR-Cas9-p19-AP1-27 and psgR-Cas9-p19-TT4-65, thereby obtaining psgR-Cas9-p19-AP1 and psgR-Cas9-p19-TT4. Both of plasmids are digested with HindIII and EcoR I, recycled, and linked into pCAMBIA1300 vector, thereby obtaining 1300-psgR-Cas9-p19-AP1 and 1300-psgR-Cas9-p19-TT4 vectors.

Analysis of Homologous Recombination-Based Transient YF-FP Report System

Homologous recombination-based transient YF-FP report system is constructed based on pA7-YFP. pA7-YFP vector can be found in FIG. 9, in which pUC18 vector is used as skeleton and a complete expression cassette of 2×35S promoter-EYFP-NOS terminator is inserted into the multiple cloning site. Two encoding sequences at 1-510 bp and 229-720 bp of YFP gene are obtained through PCR amplification by using two pairs of primers YF-FP 1F and YF-FP 1R as well as YF-FP 2F and YF-FP 2R in Table 1 and pA7-YFP vector as a template, respectively, and linked through a 18 bp cleavage linker (GGATCC ACTAGT GTCGAC) (SEQ ID NO.: 103) or a 55 bp multiple recognition sequence (MRS: ACTAGTTCCCTTTATCTCTTAGGGATAACAGGGTAATAGAGATAAAGGGAGG CCT) (SEQ ID NO.: 104), and placed into pA7-YFP vector by using XhoI/SacI to replace the original coding region of YFP. In YFP coding region of the vector, there are overlapping regions of 282 bp at both sides of cleavage linker. Protoplasts of Arabidopsis mesophyll are prepared and PEG transformation is performed according to the reported method (Yoo et al., 2007). Upon transformation, samples are cultured under darkness at room temperature for 16-24 hours, and then subject to fluorescence detection by flow cytometry.

Creation of Stable Transgenic Arabidopsis and Rice Plants

Agrobacterium GV3101 is transformed with pCambia1300 vector containing complete expression cassette of SpCas9 and complete expression cassette of chiRNA. Robust wild-type Col-0 plants during full-bloom stage are selected and subject to transgene operation through floral dip method (Clough and Bent, 1998). Transgenic plants are normally managed until seeds are harvested. Obtained seeds of T1 generation are sterilized with 5% sodium hypochlorite for 10 minutes, rinsed with sterile water for four times, and seeded on MS0 medium containing 20 μg/L of hygromycin or 50 μM kanamycin for screening. The seeds are placed at 4° C. for 2 days, transferred to a 12-hour light incubator for 10 days, and then transplanted to a 16-hour light greenhouse, and cultured. Transgenic plants are obtained by Agrobacterium-mediated transformation of calli of rice (Hiei et al., 1994).

Digestion and Sequencing Analysis of Genome Modification

Genomic DNAs of positive transformants obtained through Hygromycin-screen are extracted, PCR-amplified by using primers corresponding to target site and recovered. About 400 ng of PCR recovered product for each sample is digested by corresponding restriction enzyme overnight. Digestion reaction was analyzed by agarose gel electrophoresis (1.2-2%). Residual uncleaved stripes after digestion are recovered, linker into pZeroBack/blunt vector (TianGen Biotech, Beijing). Plasmid for monoclone is prepared by shaking, and subject to Sanger sequencing analysis by using M13F primers.

Identification of Mutant for Germline Cell Targeting

For 4 different transgenic populations of T1 generation, 32 strains are randomly selected, one leaf and one inflorescence for each population are selected after growing for two weeks and after flowering respectively, and genomic DNAs are extracted using CTAB method. Target gene fragments are PCR-amplified by using primers AP1-F133/271R, and sequenced, and for mutant, multiple signal peaks will occur from the cleavage site. For transgenic populations of T2 generation, 8 mutated strains are randomly selected, and 12 single plants are detected respectively. PCR products, sequencing results of which show multiple signal peaks, are subject to TA cloning, and 10 monoclone are picked and sequenced to determine the type of gene mutation.

Identification of Mutants Containing p19 Protein

60 strains are randomly selected for 1300-psgR-Cas9-p19-AP1/TT4 transgenic plant population of T1 generation respectively, grow for 2 weeks, and then one leaf is taken, genomic DNA of which is extracted using CTAB method. Gene fragments are PCR-amplified by using AP1-F133/271R and TT4-F159/407R primers. PCR bands are detected by electrophoresis, and produced fragments are counted to determine plant line and relevant developmental phenotypes.

In Situ Hybridization

1. Material embedding: inflorescences of transgenic plants after bolting are selected as materials, fixed with 4% paraformaldehyde for 12 hours, dehydrated with graded alcohol, transparentized with xylene and embedded in paraffin.

2. Preparation of probe: Cas9 gene is amplified with primers dCas9-F3-F/R, and the resulting fragments are digested with PstI and BamHI and ligated into pTA2 vector. The resulting vector was linearized with Sal I as DNA template, and antisense and sense Biotin labeled RNA probes (Roche, 11175025910) are in vitro transcribed by using T7 and SP6RNA polymerase, respectively. Products are digested with DNase I, subject to alkaline-lysis and purified, and dissolved in formamide for storage.

3. In situ hybridization is performe following the method reported in the literature. (Brewer P B, Heisler M G, Hejatko J, Friml J, Benkova E (2006) In situ hybridization for mRNA detection in Arabidopsis tissue sections Nat Protoc 1: 1462-1467)

Northern Hybridization

Inflorescences of a plant during flowering stage is taken, and total RNA is extracted using Trizol method (Invitrogen). 50 μg of each sample is loaded, target RNA bands are separated by using 15% PAGE gel and transferred to a nitrocellulose membrane by wet transfer method (Hybond, Amersham). UV cross-linking is performed for two minutes, and then pre-hybridization is performed in hybridization solution (DIG EASY Hyb, Roche) for 1 hour, 20 μM digoxin labeled artificial sequence probe (Invitrogen) is added, and hybridization is conducted at 42° C. overnight. The membrane is washed in 2×SSC, 0.1% SDS for two times (10 mins for each time), and in 0.1×SSC, 0.1% SDS for two times (10 mins for each time). Target bands are detected with digoxigenin detection kit (Thermo Fisher), tableted for 15 minutes, and developed under X-ray.

Realtime PCR

Extracted total RNAs of a plant are treated with DNase I (Takara) for 30 minutes. Upon phenol-chloroform purification, 5 μg is taken and subject to reverse transcription (Takara). The product is diluted at 1-fold, 1 μl is taken as template, and Realtime-PCR reaction system (Biorad) is formulated. Each sample was done in triplicate, ACTIN gene is used as internal control, wild type Col is used as control, and the relative change of gene expression is calculated with 2-ΔΔCt method.

Sequence Information

TABLE 1
Sequence information

| | | | SEQ ID |
|---|---|---|---|
| use | Primer name | Primer sequence (5′→3′) | NO.: |
| clone | YF-FP 1F | ACACGCTCGAGATGGTGAGCAAGGGCGAGG | 5 |
| | YF-FP 1R | ACACGGTCGACACTAGTGGATCCGTGGCGGATCTTGAAGTTCAC | 6 |
| | YF-FP 2F | ACACGGGATCCACTAGTGTCGACGACCACATGAAGCAGCACGAC | 7 |
| | YF-FP 2R | ACACGGAGCTCTTACTTGTACAGCTCGTC | 8 |
| | Cas9-F | TTACTCGAGATGGACTATAAGGACCACGACG | 9 |
| | Cas9-R | ATTGGATCCTTACTTTTTCTTTTTTGCCTGGC | 10 |
| | AtU6-26F | AAGCTTCGTTGAACAACGGA | 11 |
| | AtU6-26R | CGAAGGGACAATCACTACTTCG | 12 |
| | Atu6-26-85F | TTATTTTAACTTGCTATTTCTAGCTCTAAAACAGGTCTTCTC | 13 |
| | | GAAGACCCAATCACTACTTCGACTCTAGCTGTA | |
| | Atu6-26-85R | GTTAAAATAAGGCTAGTCCGTTATCAACTTGAAAAAGTGGC | 14 |
| | | ACCGAGTCGGTGCTTTTTTTGTCCCTTCGAAGGGCCTTT | |
| | OsU6-2F | GGATCATGAACCAACGGCCT | 15 |
| | OsU6-2R | AACACAAGCGACAGCGCG | 16 |
| | TPCR-OSu6F | GCCAGTGTGCTGGAATTGCCCTTGGATCATGAACCAACGGCC | 17 |
| | TPCR-OsU6R | GCTCTAAAACAGGTCTTCTCGAAGACCCACACAAGCGACAGCGCG | 18 |
| Target | YF-FP Chirna1 F | GATTGTGAACTTCAAGATCCGCCA | 19 |
| sites | YF-FP chiRNA1 R | AAACTGGCGGATCTTGAAGTTCAC | 20 |
| oligos | BRI1 Chirna1 F | GATTGTGGGTCATAACGATATCTC | 21 |
| | BRI1 chiRNA1 R | AAACGAGATATCGTTATGACCCAC | 22 |
| | BRI1 Chirna2 F | GATTGGACATACATGAGCTCCTGA | 23 |
| | BRI1 chiRNA2 R | AAACTCAGGAGCTCATGTATGTCC | 24 |
| | BRI1 Chirna3 F | GATTGTAAGAGCTGACATAGCCTG | 25 |
| | BRI1 chiRNA3 R | AAACCAGGCTATGTCAGCTCTTAC | 26 |
| | GAI Chirna 1 F | GATTGATGAGCTTCTAGCTGTTCT | 27 |
| | GAI chiRNA1 R | AAACAGAACAGCTAGAAGCTCATC | 28 |
| | ROC5 Chirna1 F | GTGTGCGGAGAACGACAGCCGGTC | 29 |
| | ROC5 chiRNA1 R | AAACGACCGGCTGTCGTTCTCCG C | 30 |
| RFLP | BRI1 1F | GAATCTCTGACGAATCTATCC | 31 |
| detection | BRI1 1R | CACTCTTTCTTCATCCCATC | 32 |
| | BRI1 2F | GATGGGATGAAGAAAGAGTG | 33 |
| | BRI1 2R | CTCATCTCTCTACCAACAAG | 34 |
| | GAI F | TGTTATTAGAAGTGGTAGTGGAGTG | 35 |
| | GAI R | AGCCGTCGCTGTAGTGGTT | 36 |
| | ROC5 F | CTTTGGGGGCCTCTTTGAC | 37 |
| | ROC5 R | ATCTGCGTGCGGCGATTC | 38 |

### Example 1

CRISPR/Cas9 of Streptococcus pyogenes SF370 was used to cause targeted double-strand breaks of DNA in Arabidopsis protoplasts.

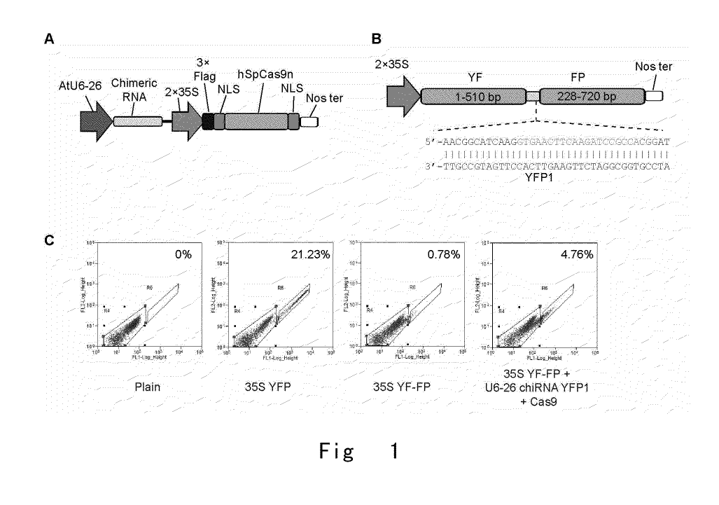

Results are shown in FIG. 1. oligo of chiRNA of YFP1 target site were constructed as YF-FP F and YF-FP R in Table 1. The results showed that when YF-FP reporter gene and CRISPR/Cas vector were co-transfected in Arabidopsis protoplasts, strong YFP signals can be obtained, and the efficiency of gene repair based on homologous recombination is up to 18.8% [(4.76%−0.78%)/21.23%]. It is showed that the constructed CRISPR/Cas system can exert its function and double strand breaks in DNA sequences can be efficiently produced in plant cells.

### Example 2

Single binary vector for Agrobacterium-mediated transformation of Arabidopsis and rice was constructed to express chiRNA and hSpCas9, and two Arabidopsis genes BRI1 and GAI as well as one rice gene ROC5 were selected to design target site.

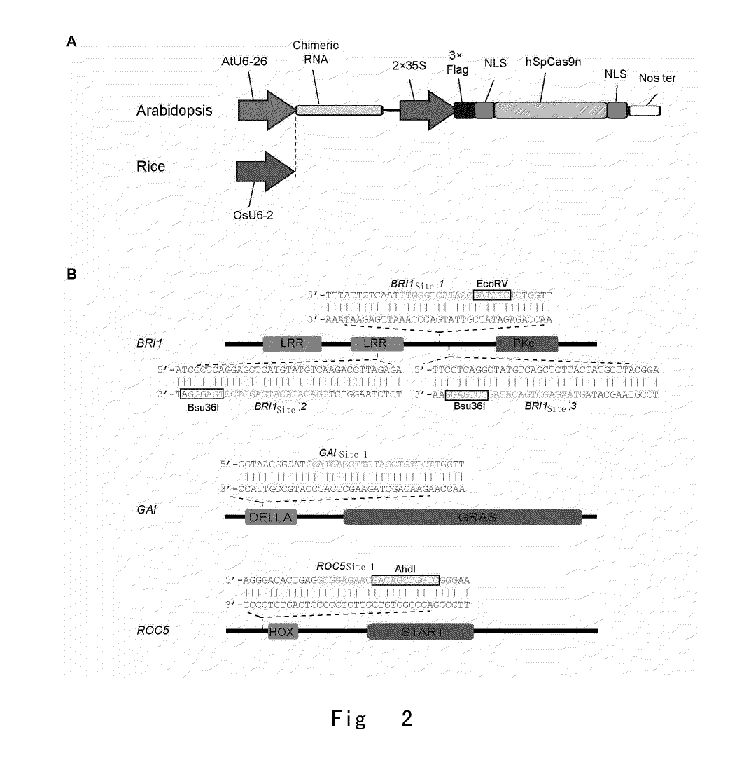

Results are shown in FIG. 2. Cas9 expression cassette in vector is identical. For chiRNA expression cassettes, AtU6-26 promoter was used for transformation of Arabidopsis, and OsU6-2 promoter was used for transformation of rice. Oligos corresponding to chiRNA constructs of BRI1 sites 1, 2, 3 were BRI1 chiRNA1 F and BRI1 chiRNA1 R, BRI1 chiRNA2 F and BRI1 chiRNA2 R, BRI1 chiRNA3 F and BRI1 chiRNA3 R, respectively. oligos corresponding to chiRNA constructs of GAI site 1 was GAI chiRNA1 F and GAI chiRNA1 R in Table 1. oligos corresponding to chiRNA constructs of ROC5 site 1 was ROC5 chiRNA1 F and ROC5 chiRNA1 R in Table 1.

### Example 3

Stable transgenic plants of Arabidopsis and rice were generated with targeted gene sites modified.

Results are shown in FIG. 3. PCR primers for identifying transgenic plants of BRI1 sites 1 and 3 by RFLP are BRI1 1F and BRI1 1R shown in Table 1, and PCR primers for identifying transgenic plants of BRI1 site 2 by RFLP are BRI1 2F and BRI1 2R shown in Table 1. PCR primers for identifying transgenic plants of GAI site 1 by RFLP are GAI F and GAI R shown in Table 1. PCR primers for identifying transgenic plants of ROC5 site 1 by RFLP are ROC5 F and ROC5 R shown in Table 1.

The results show that a large percentage of T1 transgenic Arabidopsis plants exhibit similar phenotype to homozygous mutants of the target gene locus during early growth stage. RFLP digestion analysis showed that, for target sites in certain transgenic plants, there are significantly fragments which can not be digested remained in PCR products, indicating that natural cleavage sites at target sites of some cells in these plants have been lost. Further sequencing results show that transgenic plants of T1 generation for selected target genes of Arabidopsis and rice have multiple types of DNA mutations in the target gene locus, including short deletion, insertion or replacement. It means that targeted gene cleavage can be efficiently performed by CRISPR/Cas systems in transgenic plants of Arabidopsis and rice on multiple sites of genome, thereby obtaining modifications of specific genes.

### Example 4

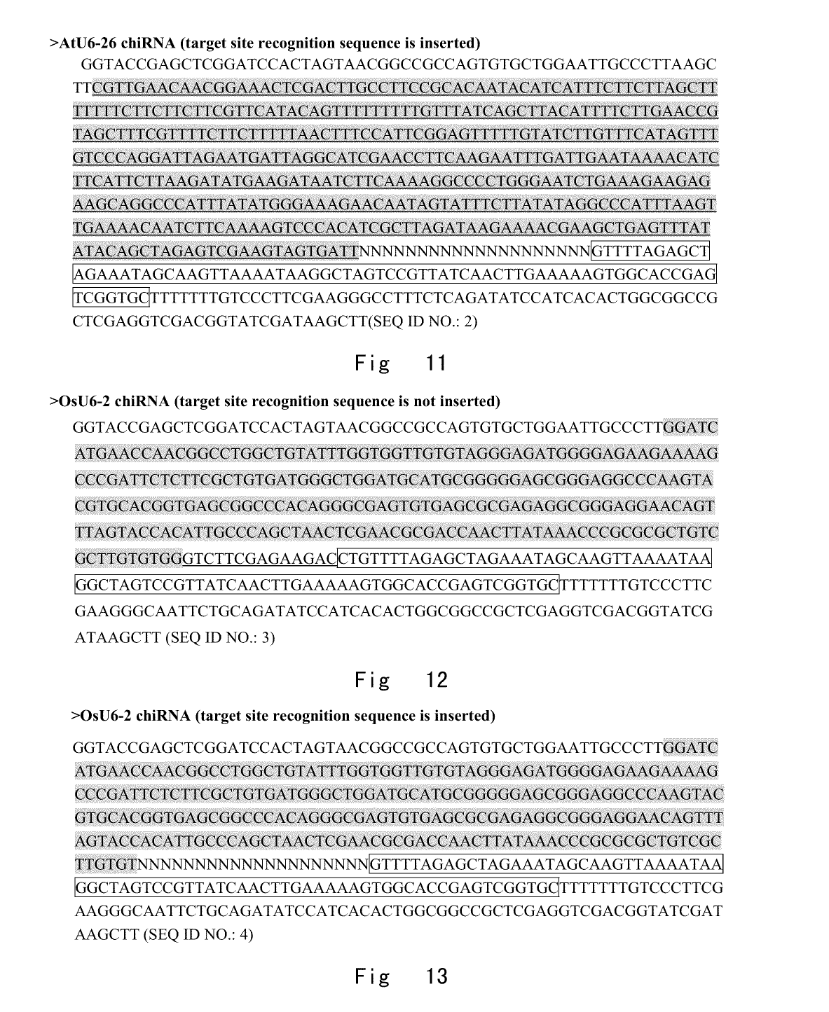

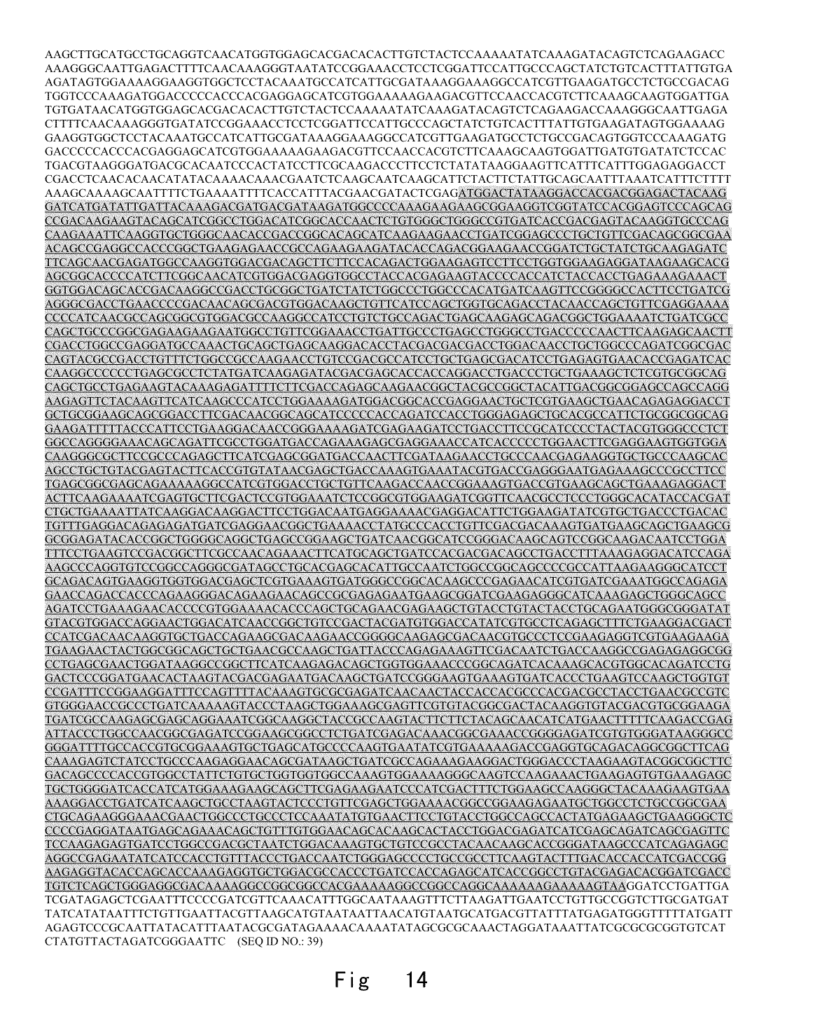

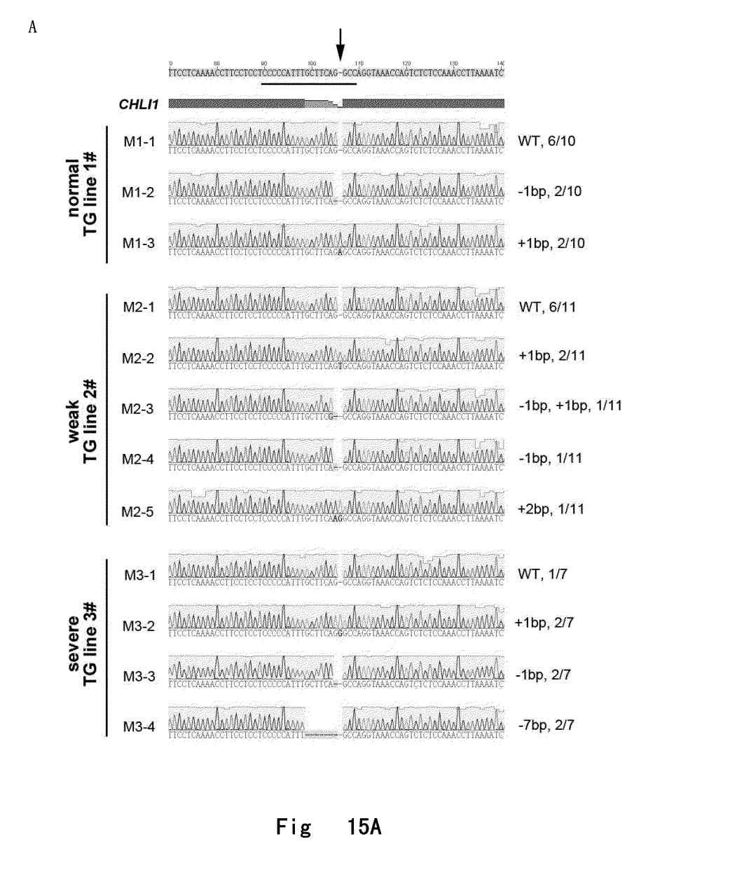

Targeted gene insertions and deletions were induced in BRI1 1 gene locus 1 of several Arabidopsis plants by using engineered chiRNA: Cas9 (FIGS. 11, 13).

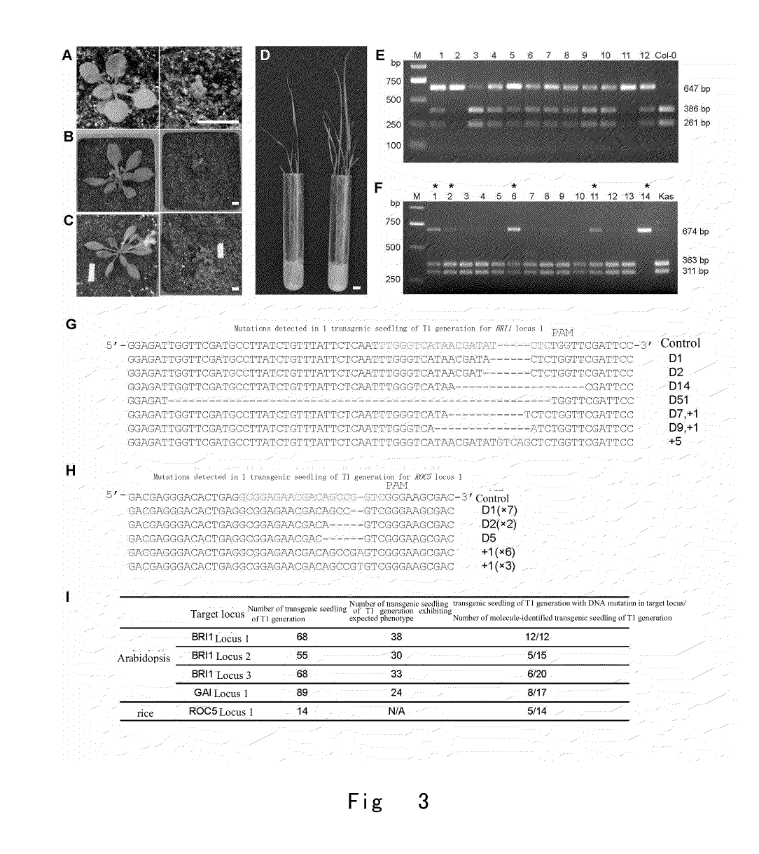

Results are shown in FIG. 4. 12 independent transgenic plants of T1 generation were sequenced and 75 mutations were detected from 98 clones, obtaining 37 different types of mutations in total. Note: there are insertion and deletion in some sequences. The results show that targeted gene cleavage can be efficiently performed by CRISPR/Cas systems in target gene locus of Arabidopsis, thereby obtaining modifications of specific genes.

### Example 5

Targeted gene insertions and deletions were induced in BRI1 2 gene locus 1 of several Arabidopsis plants by using engineered chiRNA: Cas9.

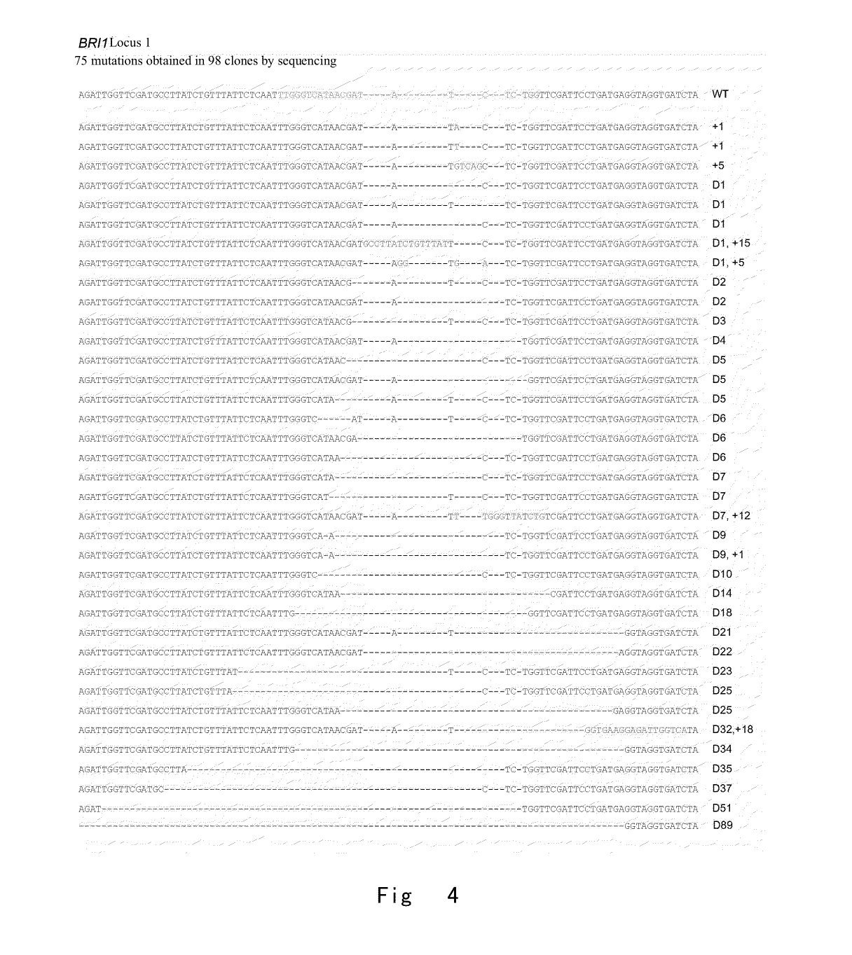

Results are shown in FIG. 5. 3 independent transgenic plants of T1 generation were sequenced and 28 mutations were detected from 71 clones, and there were 2 or more types of mutations in each plant. The results show that targeted gene cleavage can be efficiently performed by CRISPR/Cas systems in target gene locus of Arabidopsis, thereby obtaining modifications of specific genes.

### Example 6

Targeted gene insertions and deletions were induced in BRI1 2 gene locus 3 of several Arabidopsis plants by using engineered chiRNA: Cas9.

Results are shown in FIG. 6. 4 independent transgenic plants of T1 generation were sequenced and 22 mutations were detected from 34 clones, and there were 2 or more types of mutations in each plant. The results show that targeted cleavage can be efficiently performed by CRISPR/Cas systems in target gene locus of Arabidopsis, thereby obtaining modifications of specific genes.

### Example 7

Targeted gene insertions and deletions were induced in GAI gene locus 1 of Arabidopsis by using engineered chiRNA: Cas9.

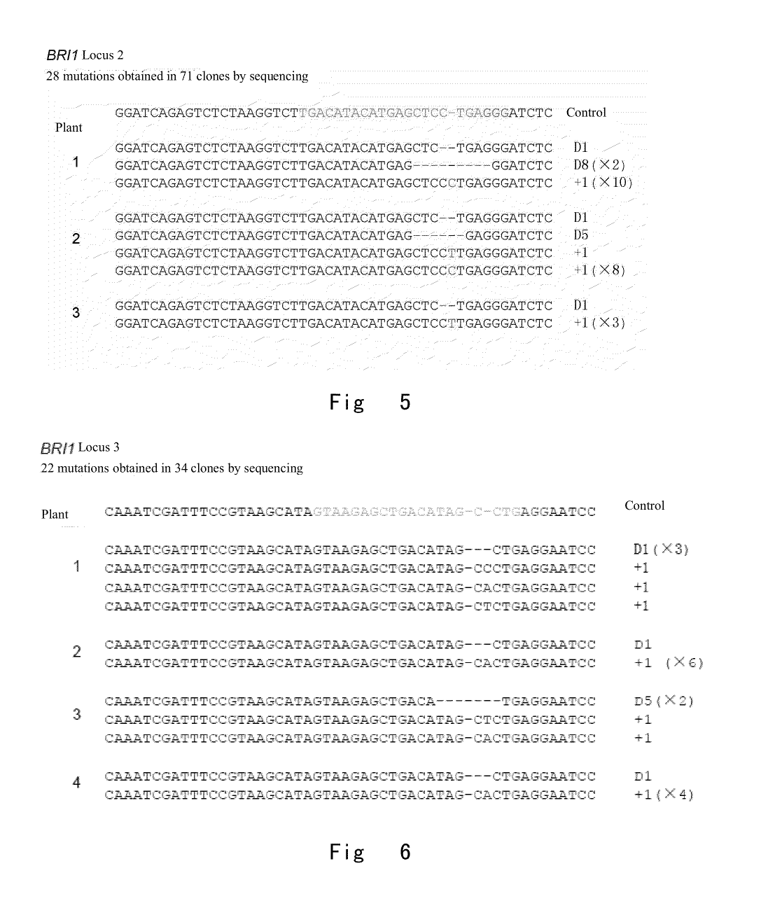

Results are shown in FIG. 7. 3 independent transgenic plants of T1 generation were sequenced and 17 mutations were detected from 53 clones, and there were one or more types of mutations in each plant. The results show that targeted cleavage can be efficiently performed by CRISPR/Cas systems in target gene locus of Arabidopsis, thereby obtaining modifications of specific genes.

### Example 8

Targeted gene insertions and deletions were induced in ROC5 gene locus 1 of rice by using engineered chiRNA: Cas9.

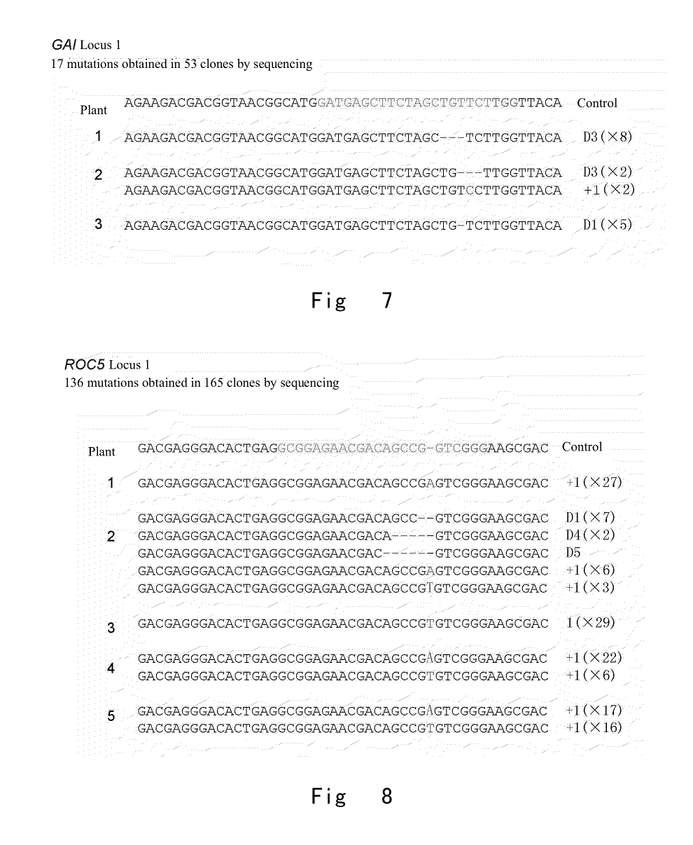

Results are shown in FIG. 8. 15 independent transgenic rice of T1 generation were sequenced and 136 mutations were detected from 165 clones, and there were one or up to 5 types of mutations in each plant. The results show that targeted cleavage can be efficiently performed by CRISPR/Cas systems in target gene locus of rice, thereby obtaining modifications of specific genes.

Summary of part of experiments of the above Examples is shown in Table 2:

TABLE 2
Statistics of mutations at target sites detected in transgenic plants
of T1 generation of Arabidopsis and rice

| | | The | The number of | The number of |
|---|---|---|---|---|
| | | number of | clones with | different types of |
| | Plant | sequenced | mutations at target | mutations at target |
| | No. | clones | site | site |
| BRI1 site 1 | 1 | 9 | 7 | 4 |
| | 2 | 8 | 8 | 8 |
| | 3 | 8 | 3 | 3 |
| | 4 | 10 | 8 | 7 |
| | 5 | 7 | 7 | 4 |
| | 6 | 8 | 5 | 4 |
| | 7 | 7 | 6 | 5 |
| | 8 | 7 | 4 | 4 |
| | 9 | 10 | 8 | 5 |
| | 10 | 10 | 9 | 6 |
| | 11 | 6 | 4 | 4 |
| | 12 | 8 | 6 | 6 |
| | total | 98 | 75 | 60 |
| BRI1 site 2 | 1 | 23 | 13 | 3 |
| | 2 | 24 | 11 | 4 |
| | 3 | 24 | 4 | 2 |
| | total | 71 | 28 | 9 |
| BRI1 site 3 | 1 | 10 | 6 | 4 |
| | 2 | 9 | 7 | 2 |
| | 3 | 6 | 4 | 3 |
| | 4 | 9 | 5 | 2 |
| | total | 34 | 22 | 11 |
| GAI site 1 | 1 | 15 | 8 | 1 |
| | 2 | 19 | 4 | 2 |
| | 3 | 19 | 5 | 1 |
| | total | 53 | 17 | 4 |
| ROC5 site 1 | 1 | 33 | 27 | 1 |
| | 2 | 27 | 19 | 5 |
| | 3 | 31 | 29 | 1 |
| | 4 | 41 | 28 | 2 |
| | 5 | 33 | 33 | 2 |
| | total | 165 | 136 | 11 |

### Example 9

Example 4 was repeated, except that, AtU6-26 was replace by promoter AtU6-1. Targeted gene insertions and deletions were induced in BRI1 1 gene locus 1 of several Arabidopsis plants by using engineered chiRNA: Cas9

10 independent transgenic rice of T1 generation were sequenced. Results showed that mutations can also be introduced into genome by using AtU6-1, while at relatively lower frequency, and is less than 10% of AtU6-26. It suggests that AtU6-26 is a particularly preferred first plant promoter.

### Example 10

Two different genes in Arabidopsis were simultaneously mutated at target sites.

P2×1300-sgr-Cas9 vector was used in several Arabidopsis plants to induce targeted gene insertions and deletions at CHLI1 and CHLI2 loci. Results are shown in FIG. 15, Table 4 and Table 5. 3 independent transgenic rice of T1 generation were sequenced and there were several types of mutations at CHLI1 and CHLI2 loci in each plant. The results show that targeted gene cleavage can be simultaneously and efficiently performed by CRISPR/Cas systems in several target gene locus of Arabidopsis, thereby obtaining modifications of several specific genes.

chiRNA oligos used in the construction of vectors are sgCHLI1-S101 and sgCHLI1-A101, as well as sgCHLI2-S280 and sgCHLI2-A280 in Table 3. PCR primers used in SURVEYOR analysis for detecting transgenic plants are CHLI1-3-F and CHLI1-262-R, as well as CHLI2-3-F and CHLI2-463-R in Table 3.

### Example 11

Simultaneous mutation and deletion of large fragment at two sites within the same gene of Arabidopsis were achieved through target sites.

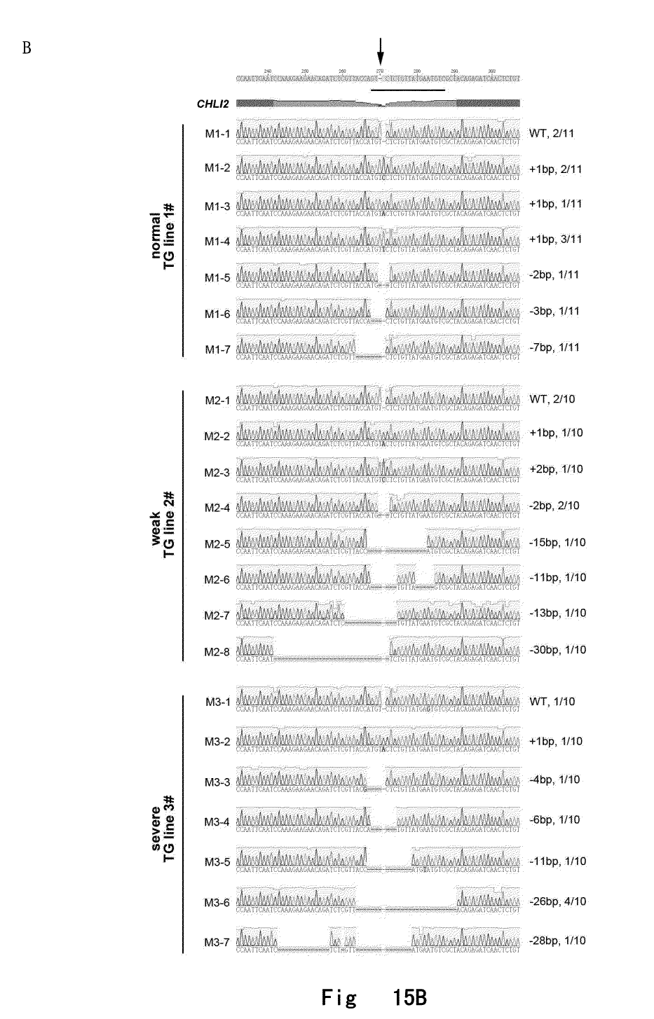

P2×1300-sgr-Cas9 vector was used in several Arabidopsis plants to induce targeted gene insertions and deletions at two sites of TT4 gene and cause deletion of large fragment between the two sites. Results are shown in FIG. 16, Table 4 and Table 5. Eleven independent transgenic rice of T1 generation were sequenced and identified, there were several types of mutations at two sites of TT4 gene in each plant, and deletion of whole sequence between the target sites was detected in several plants. The results show that targeted gene cleavage can be simultaneously and efficiently performed by CRISPR/Cas systems in several sites within the same gene of Arabidopsis, and deletion of big fragment can be achieved.

chiRNA oligos used in the construction of vectors are sgTT4-S65 and sgTT4-A65, as well as sgTT4-S296 and sgTT4-A296 in Table 3. PCR primers used in SURVEYOR analysis for detecting transgenic plants are TT4-1-F and TT4-362-R, as well as TT4-F-159 and TT4-407-R in Table 3.

TABLE 3
List of primers

| | | | SEQ ID |
|---|---|---|---|
| use | Primer name | Primer sequence (5′→3′) | NO.: |
| clone | pAtU6-F-HindIII | GCCAAGCTTCATTCGGAGTTTTTGTATCTTGTTTC | 42 |
| | pAtU6-R | AATCACTACTTCGACTCTAGCTGTATATAAACTCAGCTTCG | 43 |
| | sgR-F-U6 | CGAAGTAGTGATTGGGTCTTCGAGAAGACCTGTTTTAG | 44 |
| | sgR-R-SmaI | 5′TATCCCGGGGCCATTTGTCTGCAGAATTGGC | 45 |
| | pAtUBQ1-F-SmaI | TGGCCCCGGGATATTTCACAAATTGAACATAGACTAC | 46 |
| | pAtUBQ1-R-Cas | CCTTATAGTCCATGGTTTGTGTTTCGTCTCTCTCACGTAG | 47 |
| | Cas9-F-pUBQ | CACAAACCATGGACTATAAGGACCACGACGGAG | 48 |
| | Cas9-R-BamHI | TCTGGATCCTTACTTTTTCTTTTTTGCCTGGCCGGCC | 49 |
| | tUBQ1-F-BamHI | TAAGGATCCAGAGACTCTTATCAAGAATCCCATCTCTTGC | 50 |
| | tUBQ-R-KpnI | ACGGTACCACATAAACGGTCATTATTTCACGATACTTGTATAG | 51 |
| | pAtU6-F-KpnI | GTGGTACCCATTCGGAGTTTTTGTATCTTGTTTC | 52 |
| | sgR-EcoRI | ACGAATTCGCCATTTGTCTGCAGAATTGGC | 53 |
| | sgR-BsaI-F | GATTGGAGACCGAGGTCTCT | 70 |
| | sgR-BsaI-R | AAACAGAGACCTCGGTCTCC | 71 |
| | SPL5′-F-XmaI | TTACCCGGGAACACGAAGTCACAAAACCC | 76 |
| | SPL5′-R-BsaI | GGTCTCCCATGGTGATGATGATCTTCTTCTCGG | 77 |
| | SPL3′-F-BamHI | AATGGATCCGTTTGTTTGTTTTTTAATCGTTTTCATCAACATG | 78 |
| | SPL3′-R-KpnI | AATGGTACCACGAGAACGTGCTGAGC | 79 |
| Mutation Detection | CHLI1-3 -F | GGCGTCTCTTCTTGGAACATC | 54 |
| | CHLI1-262-R | CCGAAACATGGTAACGAGACC | 55 |
| | CHLI2-3-F | GGCGTCTCTTCTCGGAAGAT | 56 |
| | CHLI2-463-R | CGGATAAACAGGTCTTGCAC | 57 |
| | TT4-1-F | ATGGTGATGGCTGGTGCTTC | 58 |
| | TT4-362-R | CATGTAAGCACACATGTGTGGG | 59 |
| | TT4-F-159 | CTGCCCGTCCATCTAACCTAC | 60 |
| | TT4-407-R | GACTTCGACCACCACGATGT | 61 |
| | AP1-F113 | GGTTCATACCAAAGTCTGAGC | 80 |
| | AP1-271R | TCAAGTAGTCAACTTAAGGGGG | 81 |
| target site oligos | sgCHLI1-S101 | GATTGCCCCCATTTGCTTCAGGCC | 62 |
| | sgCHLI1-A101 | AAACGGCCTGAAGCAAATGGGGGC | 63 |
| | sgCHLI2-S280 | GATTGGACATTCATAACAGAGACA | 64 |
| | sgCHLI2-A280 | AAACTGTCTCTGTTATGAATGTCC | 65 |
| | sgTT4-S65 | GATTGAGAGAGCTGATGGACCTGC | 66 |
| | sgTT4-A65 | AAACGCAGGTCCATCAGCTCTCTC | 67 |
| | sgTT4-S296 | GATTGAGGCGACAAGTCGACAATT | 68 |
| | sgTT4-A296 | AAACAATTGTCGACTTGTCGCCTC | 69 |
| | sgR-AP1-S27 | GATTGGGGTAGGGTTCAATTGAAG | 72 |
| | sgR-AP1-A27 | AAACCTTCAATTGAACCCTACCC | 73 |
| | sgR-AP1-S194 | GATTGTGAAGTTACCAAGAATCAG | 74 |
| | sgR-AP1-A194 | AAACCTGATTCTTGGTAACTTCA | 75 |
| | sgR-MRS1-S | GATTGACAGGGTAATAGAGATAAA | 86 |
| | sgR-MRS1-A | AAACTTTATCTCTATTACCCTGT | 87 |
| | sgR-MRS2-S | GATTGGGGTAATAGAGATAAAGGG | 88 |
| | sgR-MRS2-A | AAACCCCTTTATCTCTATTACCC | 89 |
| Northern | Probe-sgR-1-bio | CAAGTTGATAACGGACTAGCC | 90 |
| Probe | Probe-sgR-3-bio | CTTGCTATTTCTAGCTCTAAAAC | 91 |
| | Probe-miR168-bio | TTCCCGACCTGCACCAAGCGA | 92 |
| Realtime-PCR | Cas9-RT-F | CACAAACCATGGACTATAAGGACCACGACGGAG | 93 |
| Primers | Cas9-RT-R | GATGGGGTGCCGCTCGTGCTTC | 94 |
| | p19-F | GAACGAGCTATACAAGGAAACGACGCTAGGG | 85 |
| | 2A-R-NcoI | AGTCCATGGCAGGTCCAGGGTTCTCCTC | 95 |
| | Actin-1S | TGGCATCAYACTTTCTACAA | 96 |
| | Actin-1A | CCACCACTDAGCACAATGTT | 97 |
| In situ | dCas9-F3-F | CATGGTCTCACGCCATCGTGCCTCAGAGCTTTC | 82 |
| hybridization | dCas9-F3-R | GATGGTCTCGGATCCTTACTTTTTCTTTTTTGCCTGGCCGGCC | 83 |
| Probe | Cas9-378R | GCTGAAGATCTCTTGCAGATAGCAGATCCGG | 84 |
| | p19-F | GAACGAGCTATACAAGGAAACGACGCTAGGG | 85 |

TABLE 4
Statistics of gene modification induced by CRISPR-Cas in transgenic
Arabidopsis plants of T1 generation

| | The number | | | | |
|---|---|---|---|---|---|
| | of | | The number of | The number of | Efficiency of |
| | transgenic | | transgenic | transgenic plants | simulataneous |
| | plants of T1 | | plants with | with mutations at 2 | mutation at 2 |
| Vector | generation | Target site | mutated site | sites | sites |
| 2 × sgR-CHLI1 & | 37 | CHLI1-101 | 28 | 25 | 68% |
| 2 | | CHLI2-280 | 33 | | |
| 2 × sgR-TT4 | 58 | TT4-65 | 49 | 43 | 74% |
| | | TT4-296 | 45 | | |

TABLE 5
Statistics of mutations at target sites detected in transgenic
Arabidopsis plants of T1 generation

| | | | | The number of | The number of |
|---|---|---|---|---|---|
| | | | The number of | clones with | clones with different |
| | | Plant | sequenced | mutation at | types of mutations |
| Vector | Target site | No. | clones | target site | at target site |
| 2 × | CHLI1-101 | 1 | 10 | 4 | 2 |
| sgR-CHLI1 | | 2 | 11 | 5 | 4 |
| & 2 | | 3 | 7 | 6 | 3 |
| | | total | 28 | 15 | 7 |
| | CHLI2-280 | 1 | 11 | 9 | 6 |
| | | 2 | 10 | 8 | 7 |
| | | 3 | 10 | 9 | 6 |
| | | total | 31 | 26 | 15 |
| 2 × sgR-TT4 | AtTT4-65 | 1 | 10 | 10 | 2 |
| | | 2 | 8 | 8 | 3 |
| | | 3 | 4 | 4 | 1 |
| | | 4 | 7 | 5 | 2 |
| | | 5 | 13 | 13 | 4 |
| | | 6 | 10 | 7 | 3 |
| | | 7 | 8 | 6 | 2 |
| | | 8 | 9 | 6 | 2 |
| | | 9 | 10 | 7 | 4 |
| | | 10 | 12 | 7 | 4 |
| | | 11 | 8 | 6 | 3 |
| | | total | 99 | 79 | 20 |
| | AtTT4-296 | 1 | 10 | 7 | 1 |
| | | 2 | 8 | 4 | 2 |
| | | 3 | 4 | 4 | 1 |
| | | 4 | 7 | 6 | 2 |
| | | 5 | 13 | 13 | 3 |
| | | 6 | 10 | 4 | 3 |
| | | 7 | 8 | 8 | 4 |
| | | 8 | 9 | 7 | 3 |
| | | 9 | 10 | 5 | 2 |
| | | 10 | 12 | 12 | 2 |
| | | 11 | 8 | 8 | 2 |
| | | total | 99 | 78 | 11 |

### Example 12

### Construction of Gene Targeting Vector for Plant Germline Cells

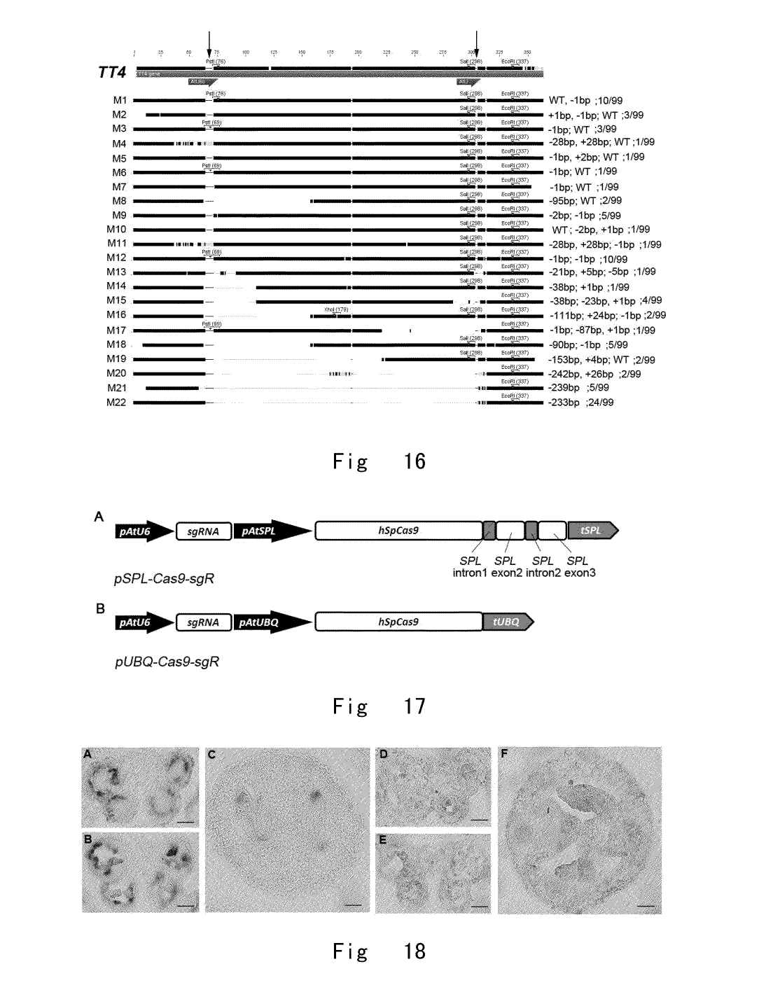

For achieving specific expression of Cas9 gene in germline cells of Arabidopsis, 3.7 Kb sequence upstream to SPL gene was cloned as promoter and 1.5 Kb downstream fragments was cloned as terminator. Humanized Cas9 gene of Streptomyces was used to replace the first exon of SPL gene, and all of the introns as well as the second and third exons of SPL gene were retained (FIG. 17, A). Meanwhile, promoter and terminator of the constitutively expressed UBQ gene were cloned to construct constitutively expressed gene targeting vector as the experimental control (FIG. 17, B).

### Example 13

### Detection of Expression Pattern of Cas9 Gene

In situ hybridization results showed that, promoter of SPL gene can drive Cas9 gene to be specifically expressed in tapetum cell (FIG. 18, A) and microspore mother cell (FIG. 18, B) during early pollen development, while UBQ promoter-driven Cas9 gene was hardly expressed in anther during the same period (FIG. 18, D, E). In addition, expression signals for SPL promoter-driven Cas9 gene can be detected in oocyte during early ovule development (FIG. 18, C). In contrast, UBQ promoter was ubiquitously expressed in ovules (FIG. 18, F). This result suggests that transcription of Cas9 gene can be specifically induced in germline cells by expression cassette of SPL gene.

### Example 14

### Detection of Mutagenesis Efficiency for Different Plant Gene Targeting Systems

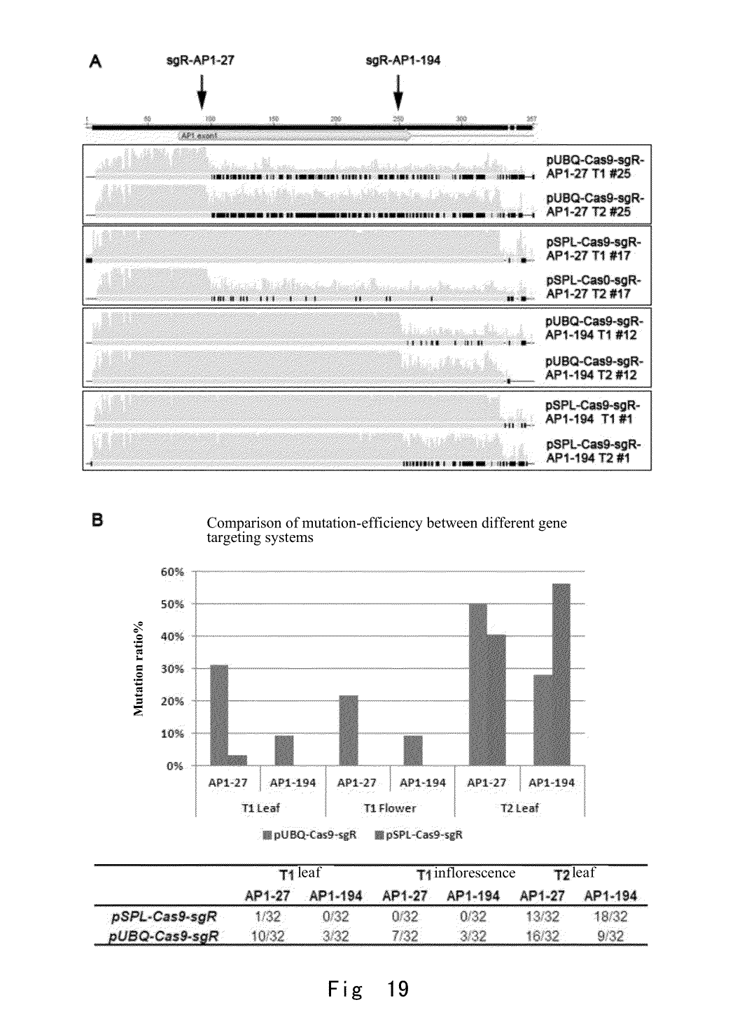

For comparing the efficiency of gene targeting between pSPL-Cas9-sgR vector and pUBQ-Cas9-sgR vector, gene targeting vectors for nucleotide site No. 27 and nucleotide site No. 194 of encoding gene of Arabidopsis APETALA (AP1) were constructed respectively, and used to transform Arabidopsis thaliana. Through PCR-amplification of the sequence of target gene and alignment of sequenced results, it was discovered that gene mutations can be detected in the plants of T1 an T2 generation for pUBQ-Cas9-sgR series of vectors, while mutations can only be detected in the transgenic population of T2 generation for pSPL-Cas9-sgR series of vectors (FIG. 19, A), which also shows that DNA cleavage activity of the vector is germline cell-specific.

According to statistics of gene targeting activities of targeting vectors during different developmental stages and in different generations, it was discovered that, firstly, there is different cleavage efficiency for different targeting sites. In terms of pUBQ-Cas9-sgR vector, the efficiency of AP1-27 was higher than that of AP1-194, whether in leaves or in inflorescences. Secondly, for some strains, mutation can be detected in leaves, however, no mutant can be produced in inflorescence. Furthermore, in the transformants of T2 generation, mutation efficiency at AP1-194 site for pSPL-Cas9-sgR was higher than AP1-27, and nearly doubled compared with pUBQ-Cas9-sgR transformant during the same period (FIG. 19, B), which means that germline cell-specific targeting vector will have good DNA cleavage activity.

### Example 15

### Statistics of Types of Mutation in Transformants of T2-Generation

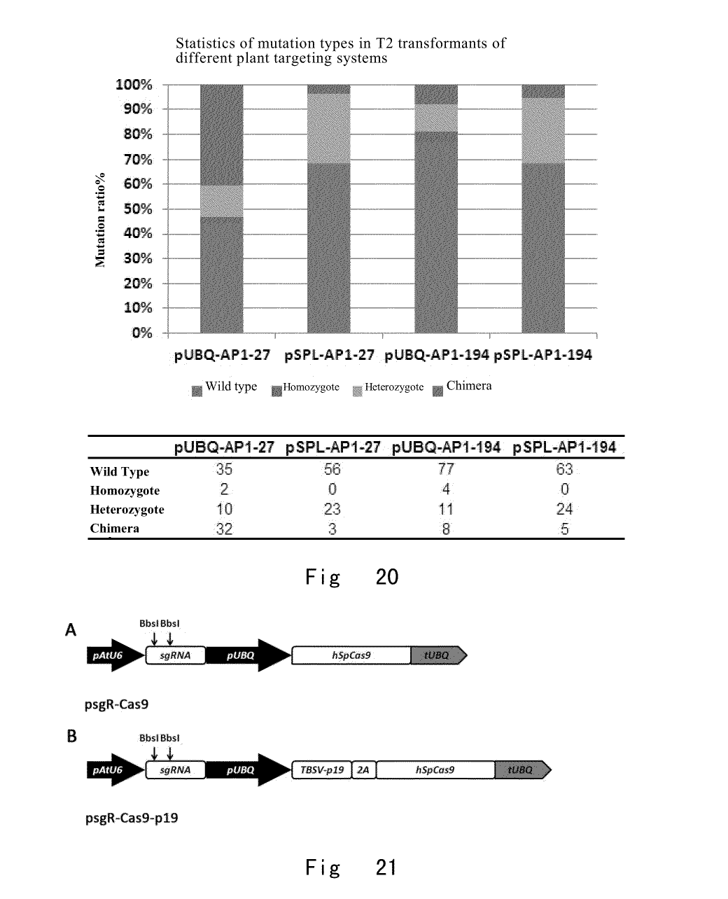

For comparing types of gene mutation produced by different gene targeting systems, 8 transgenic strains of T2 generation containing targeted gene mutation were randomly selected from 4 transgenic populations, and for each strain, 12 single plants were detected. Experimental results showed that certain percentage of homozygotes (2-4%) and heterozygous (11-12%) can be produced by constitutively expressed gene targeting system, however, chimera, genotype of which is unclear or wild type accounts for the vast majority (73%-84%). And for germline cell-specific targeting vector, about 30% of heterozygotes can be stably produced, and no homozygous plant was obtained (FIG. 20). It is speculated that SPL promoter can be expressed in male and female gametophyte, but will cause target gene mutation in one of them at a lower frequency. Of course, homozygous strain still can be isolated from T3 generation of heterozygous plants of T2 generation.

### Example 16

### Construction of Highly Efficient Gene Targeting Vector for Plants

Based on the existing gene targeting vector of Arabidopsis (FIG. 21, A), for achieving stable and efficient expression of CRISPR/Cas9 system in plants, p19 protein sequence of Tomato bushy stunt virus (TBSV) was cloned and fused in the frame of UBQ gene along with humanized Streptomyces Cas9 protein through protein cis-cutting elements 2A peptide for the transcription and translation of gene (FIG. 21, B). This fused reading frame will express two independent proteins to exert their functions respectively, due to self-splicing action of 2A peptide.

### Example 17

### Detection of Activity of Highly Efficient Targeting System for Plants

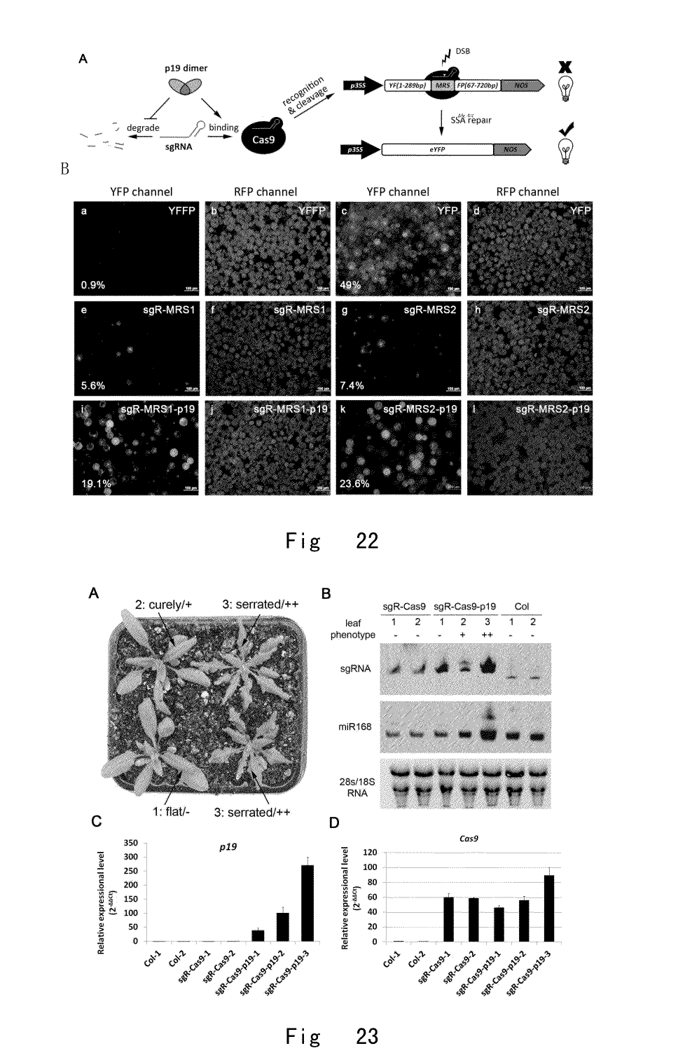

In transient expression system of Arabidopsis, protoplasts are co-transformed by CRISPR/Cas9 vector with or without p19 and YFFP reporter gene. YFFP reporter gene is the encoding gene of yellow fluorescent protein (YFP) with part of repeats, and under normal circumstances, can not be correctly expressed and translated. However, under recognition and cleavage of CRISPR/Cas9 system, double-stranded DNA breaks (DSB) will occur and endogenous DNA repair mechanism in plants will be activated to remove the repeated gene fragment, thereby producing normal and functional protein YFP (FIG. 22, A). By comparing the ratio of YFP-positive cells in two differently transfected population, experiments showed that p19 can significantly improve the gene targeting efficiency of CRISPR/Cas9 (FIG. 22, B).

### Example 18

### Expression Analysis of Highly Efficient Targeting System for Plant in Transgenic Plants

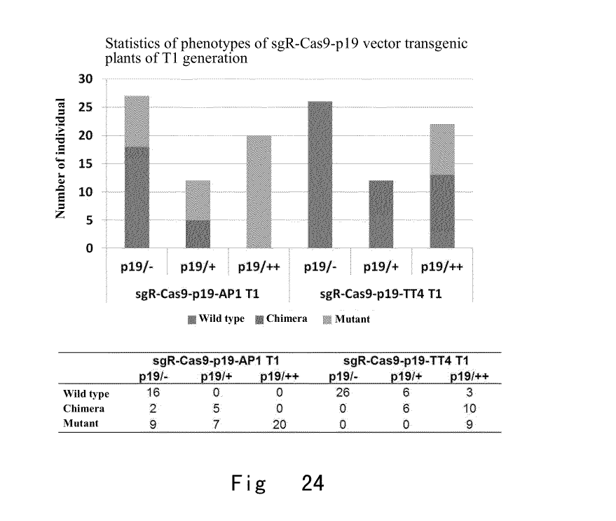

To verify the function of p19 protein in stably transformed system to improve the efficiency of plant gene targeting, two endogenous genes AP1 and TT4 in Arabidopsis was selected as target sites in this Example, and two groups of CRISPR/Cas9 gene knockout vectors with and without p19 protein were constructed, and used to transform Arabidopsis thaliana. In the obtained four transgenic populations of T1 generation, developmental phenotypes of leaves to different degree can be found. Depending on the severity of phenotype, they can be divided into three types: flat type (1/−), curl type (2/+) and serration type (3/++), and thus it is presumed that p19 protein may also interfere with miRNA-regulated leaf development process in plants (FIG. 23, A).

For verification, expression levels of sgRNA and miR168 in plants with different phenotypes were detected respectively, and it was found that the cumulative levels of sgRNA and miRNA were the highest in the plants with severe leaf phenotype (FIG. 23, B). Meanwhile, the expression level of p19 gene was also positively correlated with the severity of leaf phenotype, but hardly affected the expression of Cas9 (FIG. 23, C, D). Thus, p19 protein can indeed improve the stability of endogenous sgRNA in a plant.

### Example 19

### Functional Analysis of Highly Efficient Plant Gene Targeting System in Transgenic Plants

To understand whether p19 protein can improve targeting activity of CRISPR/Cas9 system while stabilizing sgRNA, developmental phenotype of leaves and gene mutations were recorded in two different 1300-psgR-Cas9-p19 transgenic populations, respectively.

Results showed that in both populations, about one-third of the plants exhibited severe developmental phenotype, about one-fifth of the plants exhibited slight leaf developmental phenotype. And in each population, the probability of targeted gene mutations is significantly higher in plants with leaf developmental phenotype, as compared with the plants without leaf developmental phenotype (FIG. 24), indicating that p19 can improve gene targeting efficiency of CRISPR/Cas9 system in stably transformed plants.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

### REFERENCE

1. Beumer, K., Bhattacharyya, G., Bibikova, M., Trautman, J K, and Carroll, D. (2006). Efficient gene targeting in Drosophila with zinc-finger nucleases. Genetics 172, 2391-2403.
2. Clough, S J, and Bent, A F (1998) Floral dip: A simplified method for Agrobacterium-mediated transformation of Arabidopsis thaliana The Plant Journal 16, 735-743.
3. Cui, B., Zhu, X., Xu, M., Guo, T., Zhu, D., Chen, G., Li, X., Xu, L., Bi, Y., Chen, Y., Xu, Y., Wang, W., Wang, H., Huang, W., and Ning, G. (2011). A genome-wide association study confirms previously reported loci for type 2 diabetes in Han Chinese. PLoS One 6, e22353.
4. Hiei, Y., Ohta, S., Komari, T., and Kumashiro, T. (1994). Efficient transformation of rice (Oryza sativa L.) mediated by Agrobacterium and sequence analysis of the boundaries of the T-DNA. The Plant Journal 6, 271-282.
5. Hwang, W Y, Fu, Y., Reyon, D., Maeder, M L, Tsai, S Q, Sander, J D, Peterson, R T, Yeh, J R J, and Joung, J K (2013). Efficient genome editing in zebrafish using a CRISPR-Cas system. Nat Biotech 31, 227-229.
6. Jiang, W., Bikard, D., Cox, D., Zhang, F., and Marraffini, L A (2013). RNA-guided Editing of Bacterial Genomes using CRISPR-Cas Systems. Nat Biotech 31, 233-239.
7. Jinek, M., Chylinski, K., Fonfara, I., Hauer, M., Doudna, J A, and Charpentier, E. (2012). A Programmable Dual-RNA-guided DNA endonuclease in Adaptive Bacterial Immunity. Science 337, 816-821.
8. Jinek, M., East, A., Cheng, A., Lin, S., Ma, E., and Doudna, J. (2013). RNA-programmed genome editing in human cells. ELife 2.
9. Li, L., Piatek, M., Atef, A., Piatek, A., Wibowo, A., Fang, X., Sabir, J., Zhu, J.-K., and Mahfouz, M. (2012). Rapid and highly efficient construction of TALE-based transcriptional regulators and nucleases for genome modification. Plant Molecular Biology 78, 407-416.
10. Mahfouz, M M, Li, L., Shamimuzzaman, M., Wibowo, A., Fang, X., and Zhu, J K (2011). De novo-engineered transcription activator-like effector (TALE) hybrid nuclease with novel DNA binding specificity creates double-strand breaks. Proc Natl Acad Sci USA 108, 2623-2628.
11. Mali, P., Yang, L., Esvelt, K M, Aach, J., Guell, M., DiCarlo, J E, Norville, J E, and Church, G M (2013). RNA-Guided via Cas9 Human Genome Engineering. Science 339, 823-826.
12. Meng, X., Noyes, M B, Zhu, L J, Lawson, N D, and Wolfe, S A (2008). Targeted Gene inactivation in zebrafish using Engineered Zinc-Finger nucleases. Nature Biotechnology 26, 695-701.
13. Meyer, M., de Angelis, M H, Wurst, W., and Kuhn, R. (2010). Gene targeting by homologous recombination in Mouse zygotes mediated by Zinc-Finger nucleases. Proceedings of the National Academy of Sciences of the United States of America 107, 15022-15026.
14. Meyer, M., Ortiz, O., Hrabe de Angelis, M., Wurst, W., and Kuhn, R. (2012). Modeling disease mutations by Gene targeting in one-Cell Mouse Embryos. Proceedings of the National Academy of sciences of the United States of America 109, 9354-9359.
15. Shan, Q., Wang, Y., Chen, K., Liang, Z., Li, J., Zhang, Y., Zhang, K., Liu, J., Voytas, D F, Zheng, X., and. Gao, C. (2013) Rapid and Efficient Gene Modification in Rice and Brachypodium Using TALENs Mol Plant doi: 10.1093/mp/sss162.
16. Shen, B., Zhang, J., Wu, H., Wang, J., Ma, K., Li, Z., Zhang, X., Zhang, P., and Huang, X. (2013). Generation of gene-modified mice via Cas9/RNA-mediated gene targeting. Cell Res 23, 720-723.
17. Shukla, V K, Doyon, Y., Miller, J C, DeKelver, R C, Moehle, E A, Worden, S E, Mitchell, J C, Arnold, N L, Gopalan, S., and Meng, X. (2009). Precise genome modification in the crop species Zea mays using zinc-finger nucleases. Nature 459, 437-441.
18. Wang, H., Yang, H., Shivalila, C S, Dawlaty, M M, Cheng, A W, Zhang, F., and Jaenisch, R. (2013). One-Step Generation of Mice Carrying Mutations in Multiple Genes by CRISPR/Cas-Mediated Genome Engineering. Cell.
19. Weinthal, D M, Taylor, R A, and Tzfira, T. (2013). Nonhomologous End Joining-Mediated Gene Replacement in Plant Cells. Plant Physiology 162, 390-400.
20. Yoo, S D, Cho, Y H, and Sheen, J. (2007) Arabidopsis mesophyll Protoplasts: a versatile system for transient Gene expression Cell Analysis Nature Protocols 2, 1565-1572.

1. A targeted modification method for plant genome, comprising the steps of:
(a) introducing a nucleic acid construct expressing chimeric RNA and Cas protein into a plant cell to obtain a transformed plant cell, wherein the chimeric RNA is a chimera consisting of CRISPR RNA (crRNA) specifically recognizing targeted sites to be modified (or to be cut) and trans-activating crRNA (tracrRNA); and
(b) under suitable conditions, forming chimeric RNA (chiRNA) through transcription of said nucleic acid construct in the transformed plant cell and expressing said Cas protein in said transformed plant cell, so that, in said transformed plant cell, targeted cleavage on genomic DNA is conducted by Cas protein under the guidance of said chimeric RNA, thereby performing targeted modification on genome.

2. The method according to claim 1, wherein
said nucleic acid construct comprises a first nucleic acid sub-construct and a second nucleic acid sub-construct, wherein the first nucleic acid sub-construct and a second nucleic acid sub-constructs are independent from each other, or integrated;
wherein the first nucleic acid sub-construct comprises from 5′ to 3′ the following elements:
a first plant promoter;
encoding sequence of the chimeric RNA operably linked to the first plant promoter, and the encoding sequence of the chimeric RNA is shown in formula I:
A-B (I)
wherein,
A is DNA sequence encoding CRISPR RNA (crRNAs);
B is DNA sequence encoding trans-activating crRNA (tracrRNA);
“-” represents a linkage bond or a linker sequence between A and B; wherein a complete RNA molecule is formed through transcription of the encoding sequence of the chimeric RNA, i.e., the chimeric RNA (chiRNA); and
a RNA transcription terminator;
the second nucleic acid sub-construct comprises from 5′ to 3′ the following elements:
a second plant promoter;
encoding sequence of Cas protein operably linked to the second plant promoter, and the Cas protein is a fusion protein with nuclear localization sequence (NLS sequence) at N-end, C-end or both ends; and
a plant transcription terminator.

3. The method according to claim 2, wherein there is one or more of the first nucleic acid sub-construct (for multiple sites to be cut), and is independent to the second nucleic acid sub-construct, or the first nucleic acid sub-construct and the second nucleic acid sub-construct are integrated.

4. The method according to claim 2, wherein the followings are operably linked from 5′ to 3′ between the second plant promoter and the encoding sequence of Cas protein:
the third nucleic acid sub-construct, and preferably, said third nucleic acid sub-construct is encoding sequence of p19 protein derived from Tomato bushy stunt virus (TBSV); and
self-splicing sequence, and preferably, said self-splicing sequence is encoding sequence of 2A polypeptide (SEQ ID NO.: 98).

5. The method according to claim 1, wherein the targeted modifications include:
(i) in the absence of donor DNA, performing random insertions and deletions in specific sites of the plant genome; and
(ii) in the presence of donor DNA, performing precise insertion, deletion or replacement of DNA sequence in specific sites of the plant genome using the donor DNA as a template;
preferably, the targeted modification include gene knock-out, gene knock-in (transgene) of the plant genome and regulation (up-regulation or down-regulation) of the expression level of endogenous genes.

6. The method according to claim 1, wherein the plant includes monocots, dicots and gymnosperms;
preferably, said plant includes forestry plants, agricultural plants, crops, ornamental plants.

7. The method according to claim 2, wherein the first plant promoter is RNA polymerase III-dependent promoter.

8. The method according to claim 2, wherein the second plant promoter is RNA polymerase II-dependent promoter; preferably, includes constitutively-expressed promoter and sporocyteless (SPL) promoter specifically expressed in Arabidopsis germline cell.

9. The method according to claim 1, wherein the method further comprises: said transformed plant cell is detected for mutation or modification in genome.

10. A nucleic acid construct used in targeted modification on plant genome, the nucleic acid construct comprising a first nucleic acid sub-construct and a second nucleic acid sub-construct, wherein the first nucleic acid sub-construct and the second nucleic acid sub-constructs are independent from each other, or integrated;
wherein the first nucleic acid sub-construct comprises from 5′ to 3′ the following elements:
the first plant promoter;
encoding sequence of the chimeric RNA operably linked to the first plant promoter, and the encoding sequence of the chimeric RNA is shown in formula I:
A-B (I)
wherein,
A is DNA sequence encoding CRISPR RNA (crRNAs);
B is DNA sequence encoding trans-activating crRNA (tracrRNA);
“-” represents a linkage bond or a linker sequence between A and B; wherein a complete RNA molecule is formed through transcription of the encoding sequence of the chimeric RNA, i.e., the chimeric RNA (chiRNA); and
a RNA transcription terminator;
the second nucleic acid sub-construct comprises from 5′ to 3′ the following elements:
a second plant promoter;
encoding sequence of Cas protein operably linked to the second plant promoter, and the Cas protein is a fusion protein with nuclear localization sequence (NLS sequence) at N-end, C-end or both ends; and
a plant transcription terminator.

11. The nucleic acid construct according to claim 10, wherein the first nucleic acid sub-construct and the second nucleic acid sub-construct are integrated.

12. The nucleic acid construct according to claim 10, wherein there is one or more of the first nucleic acid sub-construct (for multiple sites to be cut).

13. A vector, said vector containing the nucleic acid construct according to claim 10;
or a vector combination, wherein the vector combination comprises a first vector and a second vector, wherein the first vector contains the first nucleic acid sub-construct of the nucleic acid construct according to claim 10, and the second vector contains the second nucleic acid sub-construct of the nucleic acid construct according to claim 10.

14. A genetically engineered cell, the cell containing the vector or vector combination according to claim 13.

15. A method for producing a plant, comprising the step of regenerating the plant cell according to claim 14 into a plant.
